(19) United States
(12) Patent Application Publication
(10) Pub. No.: US 2024/0350454 A1
(43) Pub. Date: Oct. 24, 2024

(54) BACTERIAL EFFLUX PUMP INHIBITORS AND METHODS OF USE

(71) Applicants: THE REGENTS OF THE UNIVERSITY OF COLORADO, Denver, CO (US); CRESTONE, INC., Boulder, CO (US)

(72) Inventors: Corrella S. DETWEILER, Boulder, CO (US); Amy L. CROOKS, Erie, CO (US); Urs OCHSNER, Boulder, CO (US); Xicheng SUN, Boulder, CO (US); James M. GRAHAM, Boulder, CO (US); Christina WONG, Boulder, CO (US)

(21) Appl. No.: 18/294,248

(22) PCT Filed: Jun. 2, 2022
(86) PCT No.: PCT/US2022/031944

§ 371 Date: Feb. 1, 2024

Related U.S. Application Data
(60) Provisional application No. 63/228,541, filed on Aug. 2, 2021.

(51) Int. Cl. A61K 31/40; A61K 31/44; A61P 31/04; C07D 207/08

(52) U.S. Cl. CPC A61K 31/40; A61K 31/44; A61P 31/04; C07D 207/08

(57) ABSTRACT

Disclosed herein are bacterial efflux inhibitor compounds and methods of using the compounds for treating bacterial infection.

### CROSS-REFERENCE TO RELATED APPLICATIONS

This application claims priority to U.S. Application. No. 63/228,541, filed on Aug. 2, 2021. The contents of the aforementioned application are incorporated herein by reference in its entirety.

### GOVERNMENT SUPPORT

This invention was made with government support under a grant NIH R33 AI121365 awarded by National Institute of Health. The government has certain rights in the invention.

### BACKGROUND

The rise of antibiotic resistance has emphasized the need for novel antimicrobials. Although historical approaches to antibiotic discovery have yielded many crucial therapeutics, recent attempts at identifying new drugs have lagged far behind the spread of resistance. During the golden age of antibiotic discovery in the 1940s and 1950s, actinomycete extracts were screened for growth inhibition of pathogenic bacteria. This empirical platform led to identification of the major classes of antibiotics in use today. This broth-based strategy resulted in inhibitors that targeted core growth processes: translation, DNA replication, and cell wall synthesis. Although highly effective, targeting essential processes leads to strong selection for resistance. In order to focus the search for antimicrobial compounds on targets less likely to lead to resistance, the field shifted toward screening of virulence-specific processes, aided by the advent of genomics and concomitant identification of virulence-associated targets. Pharmaceutical companies have invested in high-throughput screening of synthetic chemical libraries for inhibitory activity against validated molecular targets in biochemical assays. Over the last 30 years, target-based approaches have yielded zero antibiotics for systemic use, due to a combination of meager hit identification from screens and a widespread lack of antibacterial activity in whole bacteria.

The disconnect between biochemical inhibition and antibacterial activity has been attributed to poor intracellular accumulation of small molecules in bacteria. In particular, Gram-negative bacteria contain a cell membrane, a cell wall, and an outer membrane. This cell envelope restricts penetration of amphipathic and hydrophilic substances into the cytoplasm and poses a major challenge for antibiotics.

Bacteria also use efflux pumps as a mechanism to defend against antibiotics. Efflux pumps span the periplasm between the inner and outer membranes, capture antibiotics and host antimicrobial peptides (AMPs), and export them in an energy dependent manner. When bacteria are confronted with toxic molecules, such as antibiotics, they respond by expressing higher levels of efflux pumps. Efflux pumps capture and expel antibiotics, and most antimicrobial resistant (AMR) clinical isolates have acquired extra copies of efflux pumps and/or express them at high levels. Therefore bacterial efflux pumps are a major contributor to increasing Gam-negative bacteria antibiotic resistance.

Furthermore, bacteria that survive within host cells (e.g., Salmonella enterica, Listeria monocytogenes, Staphylococcus aureus, Mycobacterium tuberculosis) are additionally protected by the host cell membrane; some pathogens that survive within vesicles are also shielded by phagosomal membranes. Even traditional antibiotics useful against extracellular pathogens are thus ineffective against intracellular microbes. For instance, aminoglycosides and β-lactams poorly accumulate within host cells and are typically ineffective. Fluoroquinolones primarily localize to the host cell cytosol, and thus are less potent against pathogens within phagosomes. Macrolides, although concentrated to high levels within cells, are typically ineffective against vesicular microbes due to inactivation at the low pH of phagolysosomes, as biochemical approaches inherently disregard cell permeability during initial screening. Thus, poor cell permeability represents a key pitfall for virulence-targeted antibacterial agents.

The present disclosure addresses these needs.

### SUMMARY

In embodiments, the disclosure provides for compounds of Formula (I)

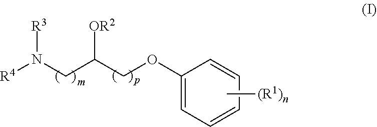

or a stereoisomer or pharmaceutically acceptable salt thereof,
   wherein:
n is 1, 2, or 3;
p is 1, 2, 3, 4, or 5;
m is 1, 2, 3, 4, or 5;
each R¹ is independently halo, alkyl, or haloalkyl;
R² is —H, alkyl, alkenyl, or alkynyl;
R³ is —H, alkyl, alkenyl or, alkynyl;
R⁴ is alkyl, alkenyl, alkynyl, cycloalkyl, or heterocyclyl, each of which is optionally substituted with one or more R⁵;
or R³ and R⁴ are taken together to form a heterocyclyl, which is optionally substituted with one or more R⁵,
R⁵ is aryl, heteroaryl, alkyl, NH₂, NHR^{A}, or NR^{A}R^{B}, or alkyl-NH₂;
R^{A} is alkyl, alkenyl, or alkynyl, each of which are optionally substituted with —OH or alkoxy; and
R^{B} is alkyl, alkenyl, or alkynyl.

In embodiments of Formula (I), R³ is —H or alkyl. In embodiments of Formula (I), R⁴ is alkyl substituted with 1 R⁵, or a heterocyclyl; and R⁵ is NH₂. In embodiments, the compounds of Formula (I) have the following structure:

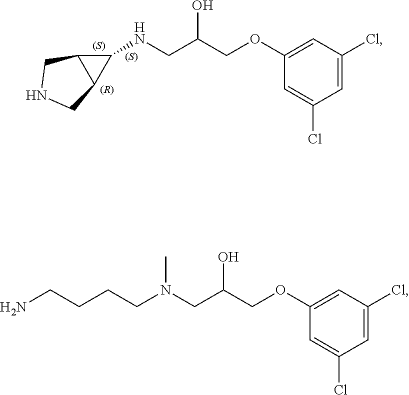

or a pharmaceutically acceptable salt thereof.

In embodiments of Formula (I), R³ and R⁴ are taken together to form a heterocyclyl, which is optionally substituted with one or more R⁵. In embodiments, the compounds of Formula (I) have a structure of Formula (II):

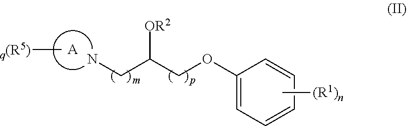

or a stereoisomer or pharmaceutically acceptable salt thereof,
   wherein:
n is 1, 2, or 3;
p is 1, 2, 3, 4, or 5;
m is 1, 2, 3, 4, or 5;
q is 1, 2, or 3;
each R¹ is independently halo, alkyl, haloalkyl;
R² is hydrogen, C₁₋₆ alkyl, C₁₋₆ alkenyl, or C₁₋₆ alkynyl;
A ring is heterocyclyl;
R⁵ is aryl, heteroaryl, alky, NH₂, NHR^{A}, or NR^{A}R^{B};
R^{A} is C₁₋₆ alkyl, C₁₋₆ alkenyl, or C₁₋₆ alkynyl, each of which are optionally substituted with —OH, or C₁₋₆ alkoxy; and
R^{B} is C₁₋₆ alkyl, C₁₋₆ alkenyl, or C₁₋₆ alkynyl, each of which are optionally substituted with —OH, or C₁₋₆ alkoxy.

In embodiments of Formula (II) the A ring is a 5-8 membered heterocyclyl, optionally having 1, 2, or 3 heteroatoms selected from N, O, and S in addition to the ring N shown in Formula (II). In embodiments, R⁵ is heteroaryl, NH₂, NHR^{A}, or NR^{A}R^{B}; R^{A} is C₁₋₆ alkyl which are optionally substituted with C₁₋₆ alkoxy; and R^{B} is C₁₋₆ alkyl.

In embodiments of Formula (I) or (II), the compounds have a structure of Formula (III):

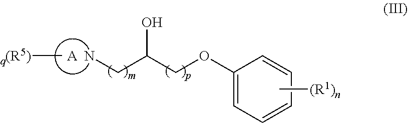

or a stereoisomer or pharmaceutically acceptable salt thereof,
   wherein:
n is 1 or 2;
p is 1, 2, or 3;
m is 1, 2, or 3;
q is 1;
each R¹ is independently halo or haloalkyl;
A ring is a 5 or 6 membered heterocyclyl;
R⁵ is heteroaryl, C₁₋₆ alkyl substituted with —NH₂, —NH₂, —NHR^{A}, or —NR^{A}R^{B};
R^{A} is C₁₋₆ alkyl which is optionally substituted with C₁₋₆ alkoxy; and
R^{B} is C₁₋₆ alkyl.

In embodiments of Formula (I), (II), or (III), the compounds have a structure of Formula (III-1) or (III-2):

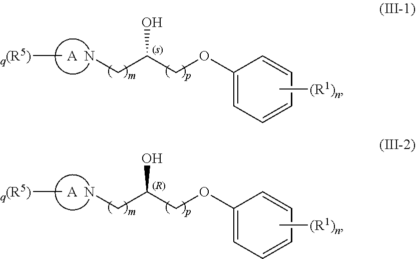

or a stereoisomer or pharmaceutically acceptable salt thereof,
   wherein n, p, m, q, R¹, A ring, and R⁵ are as defined above.

In embodiments, n is 1 or 2. In embodiments, n is 2.

In embodiments, p is 1, 2, or 3. In embodiments, p is 1.

In embodiments, m is 1, 2, or 3. In embodiments, m is 1.

In embodiments, q is 1.

In embodiments, each R¹ is independently halo or haloalkyl. In embodiments, each R¹ is independently halo. In embodiments, n is 2 and each R¹ is independently halo. In embodiments, each R¹ is Cl. In embodiments, n is 2 and each R¹ is —Cl.

In embodiments, A ring is a 5 or 6 membered heterocyclyl.

R⁵ is heteroaryl, C₁₋₆ alkyl-NH₂, —NH₂, —NHR^{A}, or —NR^{A}R^{B}. In embodiments, R^{A} is C₁₋₆ alkyl which are optionally substituted with C₁₋₆ alkoxy. In embodiments, R^{B} is C₁₋₆ alkyl.

In embodiments of Formula (I), (II), or (III), the compounds have a structure of Formula (IV):

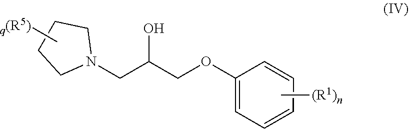

or a stereoisomer or pharmaceutically acceptable salt thereof,
   wherein:
n is 1 or 2;
q is 1;
each R¹ is Cl or fluoroalkyl;
R⁵ is alkyl substituted with —NH₂, or —NH₂, —NHR^{A}, or NR^{A}R^{B};
R^{A} is C₁₋₆ alkyl which are optionally substituted with C₁₋₆ alkoxy;
R^{B} is C₁₋₆ alkyl.

In embodiments, the compounds of Formula (I), (II), (II), or (IV) have a structure of Formula (IV-1) or (IV-2):

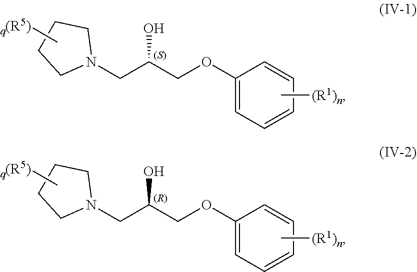

or a stereoisomer or pharmaceutically acceptable salt thereof,
   wherein n, q, R¹, and R⁵ are as defined above.

In embodiments, R⁵ alkyl substituted with —NH₂, or —NH₂. In embodiments, the compounds of Formula (IV) have the following structure:

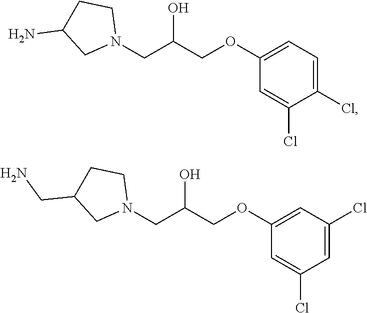 [SMILES: NC1CCN(CC(O)COc2ccc(Cl)c(Cl)c2)C1 ; NCC1CCN(CC(O)COc2cc(Cl)cc(Cl)c2)C1]

or a stereoisomer or pharmaceutically acceptable salt thereof. In embodiments, the compounds of Formula (IV) have the following structure:

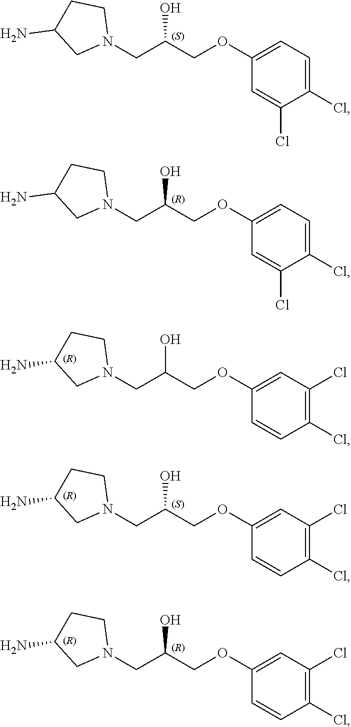

or a pharmaceutically acceptable salt thereof.

In embodiments of Formula (I), (II), or (III), the compounds have a structure of Formula (V):

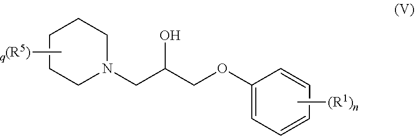

or a stereoisomer or pharmaceutically acceptable salt thereof,
   wherein:
n is 1 or 2;
q is 1;
each R¹ is Cl or fluoroalkyl;
R⁵ is heteroaryl, NH₂, NHR^{A}, or NR^{A}R^{B};
R^{A} is C₁₋₆ alkyl optionally substituted with C₁₋₆ alkoxy;
R^{B} is C₁₋₆ alkyl.

In embodiments of Formula (V), R⁵ is 5-7 membered heteroaryl having 1, 2, or 3 heteroatoms selected from N and S. In embodiments, R⁵ is 5 membered heteroaryl having 1 or 2 N heteroatoms. In embodiments, R⁵ is imidazolyl. In embodiments, the compounds of Formula (V) have the following structure:

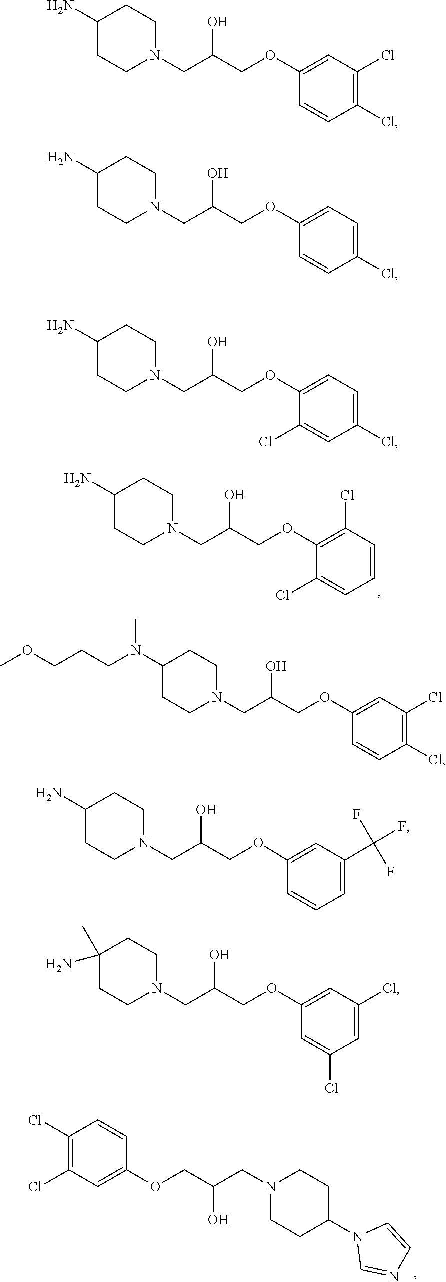

or a pharmaceutically acceptable salt thereof.

In embodiments, the disclosure provides for a pharmaceutical composition comprising therapeutically effective amount of a compound of Formula (I), (II), (III), (IV), or (V), and one or more pharmaceutically acceptable excipients.

In embodiments, the disclosure provides for a method of treating a bacterial infection in a subject in need thereof, comprising administering to the subject a pharmaceutically acceptable amount of a compound of Formula (I), (II), (III), (IV), or (V). In embodiments, the bacterial infection is caused by an intracellular pathogen. In embodiments, the bacterial infection is caused by a Gram-negative bacteria. In embodiments, the bacterial infection is caused by one or more Salmonella sp., Acinetobacter sp., Actinobacillus sp., Aeromonas sp., Bacteroide sp., Bordetella sp., Brucella sp., Burkholderia sp., Prevotella sp., Porphyromonas sp., Campylobacter sp., Citrobacter sp., Edwarsiella sp., Eikenella sp., Enterobacter sp., Escherichia sp., Francisella sp., Haemophilus sp., Helicobacter sp., Kingella sp., Klebsiella sp., Legionella sp., Moraxella sp., Morganella sp., Neisseria sp., Pasteurella sp., Plesiomonas sp., Proteus sp., Providencia sp., Pseudomonas sp., Salmonella sp., Serratia sp., Shigella sp., Stenotrophomonas sp., Streptobacillus sp., Vibrio sp., Yersinia sp., Chlamydophila sp., Ricketsia sp., Coxiella sp., Ehrlichia sp., or Bartonella sp. In embodiments, the bacterial infection is caused by one or more Salmonella species. In embodiments, the Salmonella sp. is S. enterica serovar Typhimurium. In embodiments, the bacterial infection is caused by E. coli, Klebsiella pneumonia, or Enterobacter cloacea. In embodiments, the bacterial infection is resistant to one or more antibiotics. In embodiments, the methods of the disclosure further comprise administering one or more antibiotics. In embodiments, the antibiotic is a macrolide, tetracycline, fluoroquinolone, penicillin, cephalosporin, aminoglycoside, sulfonamide, beta-lactam, tetracycline, trimethoprim-sulfamethoxazole, chloramphenicol, or lincosamide.

In embodiments, the disclosure provides for a method of inhibiting bacterial efflux pump in a subject having a bacterial infection, comprising administering to the subject a pharmaceutically acceptable amount of a compound of Formula (I), (II), (III), (IV), or (V). In embodiments, the bacterial infection is caused by an intracellular pathogen. In embodiments, the bacterial infection is caused by a Gram-negative bacteria. In embodiments, the bacterial infection is caused by one or more Salmonella sp., Acinetobacter sp., Actinobacillus sp., Aeromonas sp., Bacteroide sp., Bordetella sp., Brucella sp., Burkholderia sp., Prevotella sp., Porphyromonas sp., Campylobacter sp., Citrobacter sp., Edwarsiella sp., Eikenella sp., Enterobacter sp., Escherichia sp., Francisella sp., Haemophilus sp., Helicobacter sp., Kingella sp., Klebsiella sp., Legionella sp., Moraxella sp., Morganella sp., Neisseria sp., Pasteurella sp., Plesiomonas sp., Proteus sp., Providencia sp., Pseudomonas sp., Salmonella sp., Serratia sp., Shigella sp., Stenotrophomonas sp., Streptobacillus sp., Vibrio sp., Yersinia sp., Chlamydophila sp., Ricketsia sp., Coxiella sp., Ehrlichia sp., or Bartonella sp. In embodiments, the bacterial infection is caused by one or more Salmonella species. In embodiments, the Salmonella sp. is S. enterica serovar Typhimurium. In embodiments, the bacterial infection is caused by E. coli, Klebsiella pneumonia, or Enterobacter cloacea. In embodiments, the bacterial infection is resistant to one or more antibiotics. In embodiments, the methods of the disclosure further comprise administering one or more antibiotics. In embodiments, the antibiotic is a macrolide, tetracycline, fluoroquinolone, penicillin, cephalosporin, aminoglycoside, sulfonamide, beta-lactam, tetracycline, trimethoprim-sulfamethoxazole, chloramphenicol, or lincosamide.

In embodiments, the disclosure provides for a method of increasing sensitivity of Gram-negative bacteria to an antibiotic, comprising administering a compound of Formula (I), (II), (III), (IV), or (V) in combination with an antibiotic.

In embodiments, the disclosure provides for a method of reversing or decreasing antibiotic resistance of an antibiotic-resistant Gram-negative bacteria, comprising administering a compound of Formula (I), (II), (III), (IV), or (V).

### DETAILED DESCRIPTION

### Definitions

The term “pharmaceutically acceptable salts” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

The term “treating” means one or more of relieving, alleviating, delaying, reducing, improving, or managing at least one symptom of a condition in a subject. The term “treating” may also mean one or more of arresting, delaying the onset (i.e., the period prior to clinical manifestation of the condition) or reducing the risk of developing or worsening a condition.

The compounds of the disclosure, or their pharmaceutically acceptable salts contain at least one asymmetric center. The compounds of the disclosure with one asymmetric center give rise to enantiomers, where the absolute stereochemistry can be expressed as (R)- and (S)-, or (+) and (−). When the compounds of the disclosure have more than two asymmetric centers, then the compounds can exist as diastereomers or other stereoisomeric forms. The present disclosure is meant to include all such possible isomers, as well as their racemic and optically pure forms whether or not they are specifically depicted herein. Optically active (+) and (−) or (R)- and (S)- isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are nonsuperimposable mirror images of one another.

The term “therapeutically effective” applied to dose or amount refers to that quantity of a compound or pharmaceutical formulation that is sufficient to result in a desired clinical benefit after administration to a patient in need thereof.

The term “halo” refers to a halogen. In particular the term refers to fluorine, chlorine, bromine and iodine.

“Alkyl” or “alkyl group” refers to a fully saturated, straight or branched hydrocarbon chain group, which is attached to the rest of the molecule by a single bond. Alkyls comprising any number of carbon atoms, including but not limited to from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₈ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-Nonyl, n-decyl, n-undecyl, and n-dodecyl. Unless stated otherwise, an alkyl group can be optionally substituted.

“Alkenyl” or “alkenyl group” refers to a straight or branched hydrocarbon chain having from two to twelve carbon atoms and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise, an alkyl group can be optionally substituted.

“Alkynyl” or “alkynyl group” refers to a straight or branched hydrocarbon chain having from two to twelve carbon atoms and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise, an alkyl group can be optionally substituted.

“Alkoxy” refers to a group of the formula —ORₐ where Rₐ is an alkyl, alkenyl or alknyl as defined above containing one to twelve carbon atoms. Unless stated otherwise, an alkoxy group can be optionally substituted.

“Cycloalkyl” refers to a stable non-aromatic monocyclic or polycyclic fully saturated hydrocarbon group consisting solely of carbon and hydrogen atoms, which can include fused or bridged ring systems, having from three to twenty carbon atoms, preferably having from three to ten carbon atoms, and which is attached to the rest of the molecule by a single bond. Monocyclic cycloalkyl groups include, for example, cyclopropyl, cyclobutyl, cyclopentyl, cyclohexyl, cycloheptyl, and cyclooctyl. Polycyclic cycloalkyl groups include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Haloalkyl” refers to an alkyl group, as defined above, that is substituted by one or more halo groups, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise, a haloalkyl group can be optionally substituted.

“Aryl” refers to a hydrocarbon ring system group comprising hydrogen, 6 to 18 carbon atoms and at least one aromatic ring. For purposes of this disclosure, the aryl group can be a monocyclic, bicyclic, tricyclic or tetracyclic ring system, which can include fused or bridged ring systems. Aryl groups include, but are not limited to, aryl groups derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise, the term “aryl” is meant to include aryl groups that are optionally substituted.

“Heterocyclyl,” “heterocyclic ring” or “heterocycle” refers to a stable 3- to 20-membered ring group which consists of two to twelve carbon atoms and from one to six heteroatoms selected from the group consisting of nitrogen, oxygen and sulfur. The heterocyclyl group can be a monocyclic, bicyclic, tricyclic or tetracyclic ring system, which can include fused or bridged ring systems. The nitrogen, carbon or sulfur atoms in the heterocyclyl group can be optionally oxidized, the nitrogen atom can be optionally quaternized. The heterocyclyl group can be partially or fully saturated. Examples of such heterocyclyl groups include, but are not limited to, dioxolanyl, decahydroisoquinolyl, imidazolinyl, imidazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise, a heterocyclyl group can be optionally substituted.

“Heteroaryl” refers to a 5- to 20-membered ring system group comprising hydrogen atoms, one to thirteen carbon atoms, one to six heteroatoms selected from the group consisting of nitrogen, oxygen and sulfur, and at least one aromatic ring. For purposes of this disclosure, the heteroaryl group can be a monocyclic, bicyclic, tricyclic or tetracyclic ring system, which can include fused or bridged ring systems; and the nitrogen, carbon or sulfur atoms in the heteroaryl group can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise, a heteroaryl group can be optionally substituted.

“The term “substituted” used herein means any of the above groups wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing groups can also be optionally substituted with one or more of the above groups.

### Compounds

In embodiments, the disclosure provides for compounds that can be used to treat bacterial infections. In embodiments, compounds inhibit efflux pumps, which reduces the ability of bacteria to expel antibiotics. Thus, by inhibiting efflux pumps, the compounds of the disclosure also increase bacterial sensitivity to antibiotics. Accordingly, in embodiments, the compounds may be used in combination with antibiotics to treat antibiotic-resistant bacteria, to reduce the ability of bacteria to develop resistance to an antibiotic, or to increase sensitivity of bacteria to an antibiotic.

In some embodiments, the disclosure provides for compounds of Formula (I)

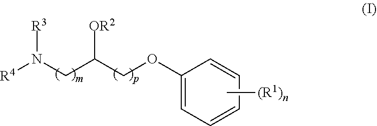

or a stereoisomer or pharmaceutically acceptable salt thereof.

In embodiments, n is 1, 2, or 3.

In embodiments, p is 1, 2, 3, 4, or 5.

In embodiments, m is 1, 2, 3, 4, or 5.

In embodiments, each R¹ is independently halo, alkyl, haloalkyl. In embodiments, alkyl is substituted. In embodiments, alkyl is unsubstituted.

In embodiments, R² is —H, alkyl, alkenyl, or alkynyl. In embodiments, alkyl is substituted. In embodiments, alkyl is unsubstituted. In embodiments, alkenyl is substituted. In embodiments, alkenyl is unsubstituted. In embodiments, alkynyl is substituted. In embodiments, alkynyl is unsubstituted.

In embodiments, R³ is —H, alkyl, alkenyl, or alkynyl. In embodiments, alkyl is substituted. In embodiments, alkyl is unsubstituted. In embodiments, alkenyl is substituted. In embodiments, alkenyl is unsubstituted. In embodiments, alkynyl is substituted. In embodiments, alkynyl is unsubstituted.

In embodiments, R⁴ is alkyl, alkenyl, alkynyl, cycloalkyl, or heterocyclyl, each of which is optionally substituted with one or more R⁵. In embodiments, alkyl is substituted. In embodiments, alkyl is unsubstituted. In embodiments, alkenyl is substituted. In embodiments, alkenyl is unsubstituted. In embodiments, alkynyl is substituted. In embodiments, alkynyl is unsubstituted. In embodiments, cycloalkyl is substituted. In embodiments, cycloalkyl is unsubstituted. In embodiments, heterocyclyl is substituted. In embodiments, heterocyclyl is unsubstituted.

In embodiments, R³ and R⁴ are taken together to form a heterocyclyl, which is optionally substituted with one or more R⁵.

In embodiments, R⁵ is aryl, heteroaryl, alkyl, NH₂, NHR^{A}, or NR^{A}R^{B}, or alkyl substituted with —NH₂. In embodiments, R^{A} is alkyl, alkenyl, or alkynyl, each of which are optionally substituted with —OH or alkoxy. In embodiments, R^{B} is alkyl, alkenyl, or alkynyl. In embodiments, alkyl is substituted. In embodiments, alkyl is unsubstituted. In embodiments, alkenyl is substituted. In embodiments, alkenyl is unsubstituted. In embodiments, alkynyl is substituted. In embodiments, alkynyl is unsubstituted.

In embodiments, n is 1, 2, or 3. In embodiments, n is 1 or 2. In embodiments, n is 1. In embodiments, n is 2. In embodiments, each R¹ is independently halo, alkyl, or haloalkyl. In embodiments, each R¹ is independently halo or haloalkyl. In embodiments, n is 2, and each R¹ is independently halo. In embodiments, n is 1 and R¹ is haloalkyl. In embodiments, haloalkyl is a C₁-C₆ alkyl substituted with 1, 2, 3, or more fluorine. In embodiments, haloalkyl is —CF₃. In embodiments, p is 1, 2, 3, 4, or 5. In embodiments, p is 1.

In embodiments, m is 1, 2, 3, 4, or 5. In embodiments, m is 1.

In embodiments, R² is —H, alkyl, alkenyl, or alkynyl. In embodiments, R² is —H.

In embodiments, R³ is —H, alkyl, alkenyl, or alkynyl. In embodiments, R³ is —H or alkyl. In embodiments, R³ is —H. In embodiments, R³ is alkyl. In embodiments, R³ is C₁-C₆ alkyl. In embodiments, R³ is methyl or ethyl. In embodiments, R³ is methyl.

In embodiments, R⁴ is alkyl, alkenyl, alkynyl, cycloalkyl, heterocyclyl, each of which is optionally substituted with one or more R⁵. In embodiments, R⁴ is alkyl or heterocyclyl, each of which is optionally substituted with one or more R⁵. In embodiments, R⁴ is C₁-C₆ alkyl. In embodiments, R⁴ is methyl, ethyl, propyl (e.g., n-propyl, i-propyl, sec-propyl) or butyl (e.g., n-butyl, i-butyl, sec-butyl, t-butyl). In embodiments, R⁴ is propyl. In embodiments, R⁴ is butyl. In embodiments, R⁵ is alkyl-NH₂. In embodiments, R⁵ is C₁-C₆ alkyl (i.e., methyl, ethyl, n-propyl, i-propyl, sec-propyl, n-butyl, i-butyl, sec-butyl, t-butyl, n-pentyl, 1-amyl, or n-hexyl) substituted with —NH₂. In embodiments, R⁵ is C₄ alkyl-NH₂. In embodiments, R⁵ is C₁—NH₂.

In embodiments, R⁴ is 5-7 membered heterocyclyl having 1, 2, or 3 heteroatoms selected from O, N, or S, and which is optionally substituted with R⁵. In embodiments, R⁴ is 5-6 membered heterocyclyl. In embodiments, R⁴ is 5-6 membered bicyclic heterocyclyl. In embodiments, R⁴ is 6 membered heterocyclyl having 1 N heteroatom. In embodiments, R⁴ is 5, 3 fused heterocyclyl having 1 N heteroatom.

In embodiments, R³ is —H or alkyl (e.g., C₁₋₃ alkyl), and R⁴ is alkyl or heterocyclyl, which is optionally substituted with 1 R⁵. In embodiments, R⁵ is —NH₂. In embodiments, the compounds have one of the following structures:

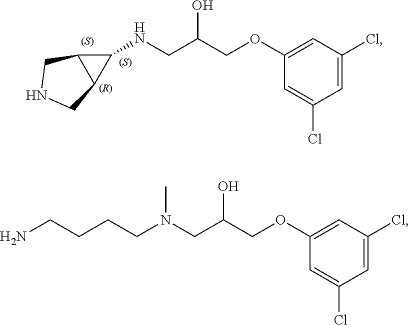

or a pharmaceutically acceptable salt thereof.

In embodiments when R³ and R⁴ are taken together to form a heterocyclyl. In embodiments, the heterocyclyl may be 5-8 membered heterocyclyl, which is optionally substituted with one or more R⁵. In embodiments, R³ and R⁴ are taken together to form 5 membered heterocyclyl, which is optionally substituted with —NH₂ or alkyl-NH₂. In embodiments, R³ and R⁴ are taken together to form 5 membered heterocyclyl, which is substituted with —NH₂ or C₁-C₆ alkyl-NH₂ (e.g., —CH₂—NH₂). In embodiments, R³ and R⁴ are taken together to form 6 membered heterocyclyl, which is optionally substituted with one or more R⁵. In embodiments, R³ and R⁴ are taken together to form 6 membered heterocyclyl, which is substituted with one or more R⁵, and R⁵ is alkyl, —NH₂, or —NR^{A}R^{B}, wherein R^{A} is alkyl optionally substituted with alkoxy and R^{B} is alkyl. In embodiments, R³ and R⁴ are taken together to form 6 membered heterocyclyl, and R⁵ is C₁-C₆ alkyl, —NH₂, or —NR^{A}R^{B}, wherein R^{A} is C₁-C₆ alkyl substituted with C₁-C₆ alkoxy and R^{B} is C₁-C₆ alkyl. In embodiments, R³ and R⁴ are taken together to form 6 membered heterocyclyl, which is substituted with R⁵, and R⁵ is —NH₂, or —NR^{A}R^{B}, wherein R^{A} is C₂-C₆ alkyl substituted with C₁-C₃ alkoxy and R^{B} is C₁-C₃ alkyl.

In embodiments, R⁵ is aryl, heteroaryl, alkyl, —NH₂, —NHR^{A}, or —NR^{A}R^{B}, or alkyl-NH₂. In embodiments, R⁵ is 6 or 8 membered aryl, 5-8 membered heteroaryl, C₁-C₆ alkyl, —NH₂, —NHR^{A}, or —NR^{A}R^{B}, or C₁-C₆ alkyl substituted with —NH₂. In embodiments, R⁵ is a 5 membered heteroaryl. In embodiments, R⁵ is imidazolyl. In embodiments, R³ and R⁴ are taken together to form 6 membered heterocyclyl, and R⁵ is a 5 membered heteroaryl (e.g., imidazolyl).

In embodiments, R^{A} is alkyl, alkenyl, alkynyl, each of which are optionally substituted with —OH, or alkoxy. In embodiments, R^{A} is C₁-C₆ alkyl. In embodiments, R⁴ is methyl. In embodiments, R⁴ is ethyl. In embodiments, R⁴ is propyl. R^{A} is C₁-C₆ alkyl substituted with C₁-C₆ alkyloxy. In embodiments, R⁴ is C₁-C₆ alkyl substituted with methoxy. In embodiments, R⁴ is propyl substituted with methoxy.

In embodiments, R^{B} is alkyl, alkenyl, alkynyl, each of which are optionally substituted with —OH, or alkoxy. In embodiments, R^{B} is C₁-C₆ alkyl. In embodiments, R^{B} is methyl.

In embodiments, the compound of Formula (I) has a structure of Formula (II):

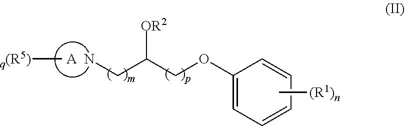

or a stereoisomer or pharmaceutically acceptable salt thereof.

In embodiments, n is 1, 2, or 3. In embodiments, n is 1. In embodiments, n is 2.

In embodiments, p is 1, 2, 3, 4, or 5. In embodiments, p is 1. In embodiments, m is 1, 2, 3, 4, or 5. In embodiments, m is 1.

In embodiments, q is 1, 2, or 3. In embodiments, q is 1. In embodiments, q is 2.

In embodiments, each R¹ is independently halo, alkyl, or haloalkyl. In embodiments, each R¹ is independently halo or haloalkyl. In embodiments, n is 2, and each R¹ is independently halo. In embodiments, n is 2, and each R¹ is independently —Cl. In embodiments, n is 1 and R¹ is haloalkyl. In embodiments, haloalkyl is a C₁-C₆ alkyl substituted with 1, 2, 3, or more fluorine. In embodiments, haloalkyl is —CF₃.

In embodiments, R² is hydrogen, C₁₋₆ alkyl, C₁₋₆ alkenyl, or C₁₋₆ alkynyl. In embodiments, R² is hydrogen.

In embodiments, A ring is heterocyclyl. In embodiments, A ring is a 5-8 membered heterocyclyl, optionally having 1, 2, or 3 heteroatoms selected from N, O, or S in addition to the ring N shown in Formula (II). In embodiments, A ring is a 5-8 membered heterocyclyl.

In embodiments, R⁵ is aryl, heteroaryl, alky, NH₂, NHR^{A}, or NR^{A}R^{B} In embodiments, R⁵ is NH₂. In embodiments, R⁵ is methyl, ethyl, or propyl (n-propyl, i-propyl, sec-propyl). In embodiments, R⁵ is methyl.

In embodiments, R^{A} is C₁₋₆ alkyl, C₁₋₆ alkenyl, C₁₋₆ alkynyl, each of which are optionally substituted with halo, OH, C₁₋₆ alkoxy. In embodiments, R^{B} is C₁₋₆ alkyl, C₁₋₆ alkenyl, C₁₋₆ alkynyl, each of which are optionally substituted with halo, OH, C₁₋₆ alkoxy. In embodiments, R⁵ is heteroaryl, NH₂, NHR^{A}, or NR^{A}R^{B}. In embodiments, R^{A} is C₁₋₆ alkyl optionally substituted with C₁₋₆ alkoxy. In embodiments, R^{A} is propyl substituted with methoxy. In embodiments, R^{B} is C₁₋₆ alkyl. In embodiments, R^{B} is methyl or ethyl. In embodiments, R^{B} is methyl.

In embodiments, the compounds of Formula (I) or (II) have a structure of Formula (III):

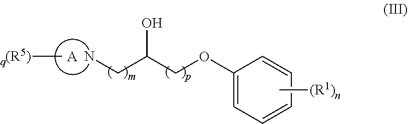

or a stereoisomer or pharmaceutically acceptable salt thereof.

In embodiments, the compound of Formula (I), (II), or (III) has a structure of Formula (III-1) or (III-2):

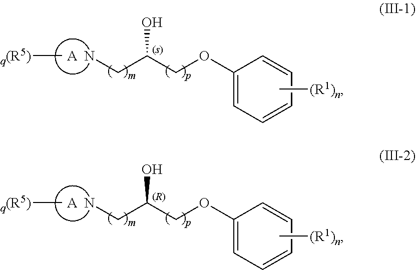

or a stereoisomer or pharmaceutically acceptable salt thereof.

In embodiments, n is 1 or 2. In embodiments, n is 2.

In embodiments, p is 1, 2, or 3. In embodiments, p is 1.

In embodiments, m is 1, 2, or 3. In embodiments, m is 1.

In embodiments, q is 1.

In embodiments, each R¹ is independently halo or haloalkyl. In embodiments, each R¹ is independently halo. In embodiments, n is 2 and each R¹ is independently halo. In each R¹ is Cl. In embodiments, n is 2 and each R¹ is independently —Cl.

In embodiments, A ring is a 5 or 6 membered heterocyclyl.

In embodiments, R⁵ is heteroaryl, C₁₋₆ alkyl, C₁₋₆ alkyl-NH₂, —NH₂, —NHR^{A}, or —NR^{A}R^{B}. In embodiments, R⁵ is —NH₂. In embodiments, R⁵ is C₁₋₆ alkyl (e.g., —CH₃) and —NH₂. In embodiments, R^{A} is C₁₋₆ alkyl optionally substituted with C₁₋₆ alkoxy. In embodiments, R^{A} is propyl substituted with methoxy. In embodiments, R^{B} is C₁₋₆ alkyl. In embodiments, R^{B} is methyl.

In embodiments, the compounds of Formula (I), (II), or (III) have a structure of Formula (IV):

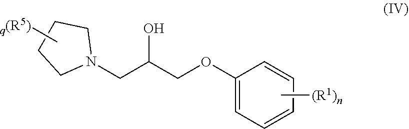

or a stereoisomer or pharmaceutically acceptable salt thereof.

In embodiments, the compounds of Formula (I), (II), (III), or (IV) has a structure of Formula (IV-1) or (IV-2):

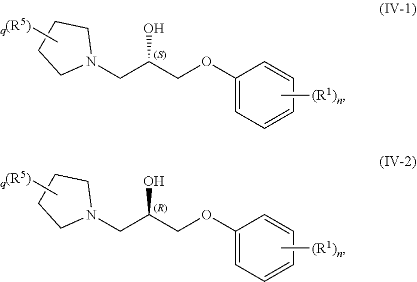

or a stereoisomer or pharmaceutically acceptable salt thereof.

In embodiments, n is 1 or 2. In embodiments, each R¹ is Cl or fluoroalkyl. In embodiments, n is 2 and each R¹ is Cl. In embodiments, n is 1 and R¹ is independently —CF₃.

In embodiments, q is 1. In embodiments, R⁵ is alkyl substituted with —NH₂, or —NH₂, —NHR^{A}, or NR^{A}R^{B}. In embodiments, R^{A} is C₁₋₆ alkyl which are optionally substituted with C₁₋₆ alkoxy. In embodiments, R^{B} is C₁₋₆ alkyl. In embodiments, R⁵ alkyl substituted with —NH₂, or —NH₂.

In embodiments, the compounds of Formula (IV) have a structure of Formula (IV.A) or (IV.B):

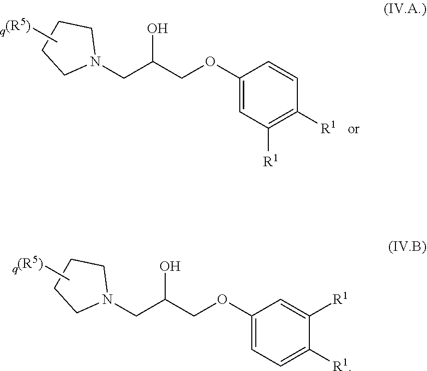

In embodiments, the compounds of Formula (IV) have the following structure:

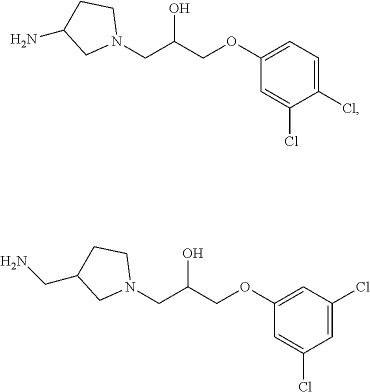 [SMILES: NC1CCN(CC(O)COc2ccc(Cl)c(Cl)c2)C1 ; NCC1CCN(CC(O)COc2cc(Cl)cc(Cl)c2)C1]

or a stereoisomer or pharmaceutically acceptable salt thereof.

In embodiments, the compounds of Formula (IV) have the following structure:

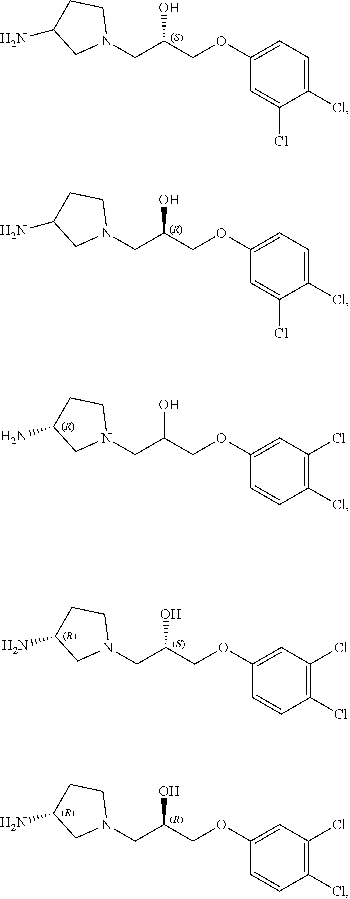

or a pharmaceutically acceptable salt thereof.

In embodiments, the compounds of Formula (I), (II), or (II), have a structure of Formula (V):

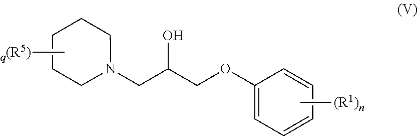

or a stereoisomer or pharmaceutically acceptable salt thereof,

In embodiments, n is 1 or 2. In embodiments, each R¹ is Cl or fluoroalkyl. In embodiments, n is 2 and each R′ is Cl. In embodiments, n is 1 and R¹ is fluoroalkyl. In embodiments, fluoroalkyl is C₁-C₆ alkyl substituted with 1, 2, 3, or more fluorine. In embodiments, fluoroalkyl is —CF₃. In embodiments, q is 1. In embodiments, R⁵ is heteroaryl, NH₂, NHR^{A}, or NR^{A}R^{B}. In embodiments, R^{A} is C₁₋₆ alkyl which is optionally substituted with C₁₋₆ alkoxy. R^{B} is C₁₋₆ alkyl. R^{A} is C₁₋₆ alkyl substituted with C₁₋₆ alkoxy. In embodiments, R^{A} is C₁₋₆ alkyl substituted with methoxy. In embodiments, R^{A} is propyl or butyl substituted with —OCH₃. In embodiments, R^{B} is C₁₋₆ alkyl. In embodiments, R^{B} is —CH₃.

In embodiments, R⁵ is 5-7 membered heteroaryl having 1, 2, or 3 heteroatoms selected from N and S. In embodiments, R⁵ is 5 membered heteroaryl having 1 or 2 N heteroatoms. In embodiments, R⁵ is imidazolyl.

In embodiments, the compounds of Formula (V) have a structure of Formula (V.A)-(V.F):

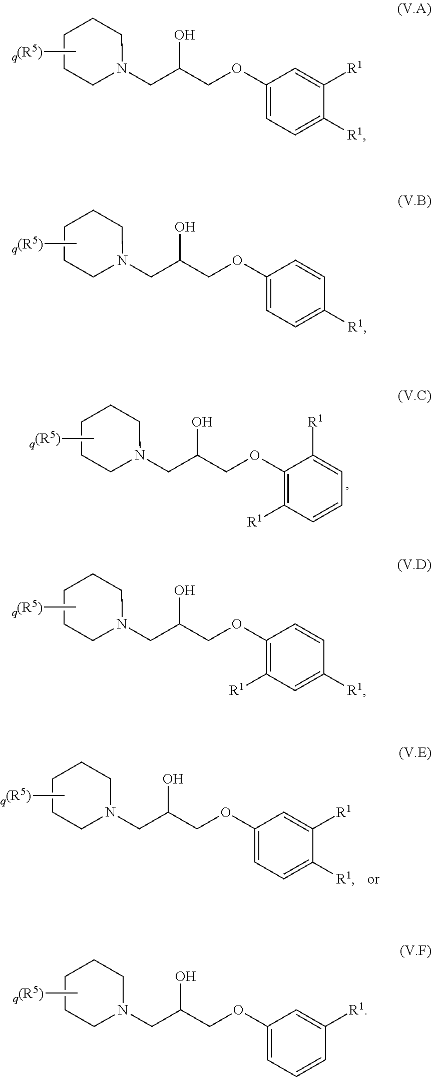

In embodiments, the compounds of Formula (V) have following structure:

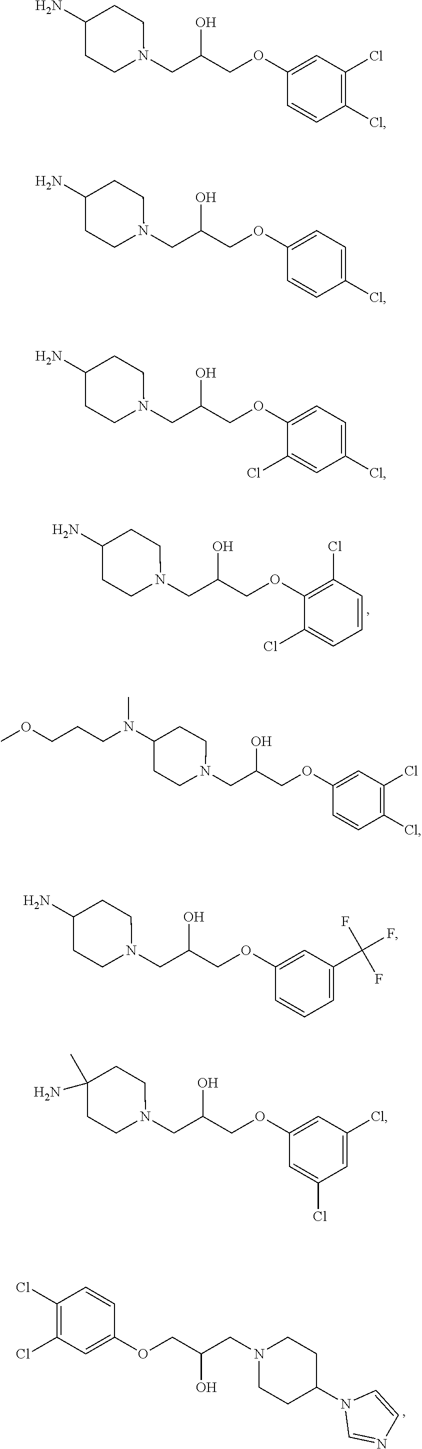

or a pharmaceutically acceptable salt thereof.

### General Synthesis

Compounds disclosed herein, including salts thereof, can be prepared using known organic synthesis techniques and can be synthesized according to any of numerous possible synthetic routes. Those skilled in the art of synthetic organic chemistry will appreciate that the selection of the starting material, and reagents will partially depend on the desired product and/or the reagents used, e.g., as various mechanisms require a primary or secondary alcohol.

The reaction for preparing compounds disclosed herein can be carried out in suitable solvents which can be readily selected by one of skill in the art of organic synthesis. Suitable solvents can be substantially non-reactive with the starting materials, the intermediates, or products at the temperatures at which the reactions are carried out, e.g., temperatures which can range from room temperature to the solvent's boiling temperature. The selection of appropriate protecting group, can be readily determined by one skilled in the art. A given reaction can be carried out in one solvent or mixture of solvents.

In embodiments, the compounds disclosed herein can be prepared by following Schemes I-IV.

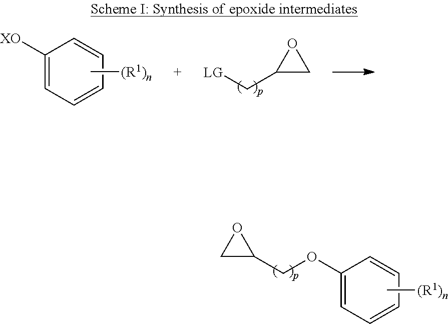

wherein:
X is H or a cation;
LG is a leaving group, such as a halide;
n is 1, 2, or 3;
p is 1, 2, 3, 4, or 5;
each R¹ is independently halo, alkyl, haloalkyl.

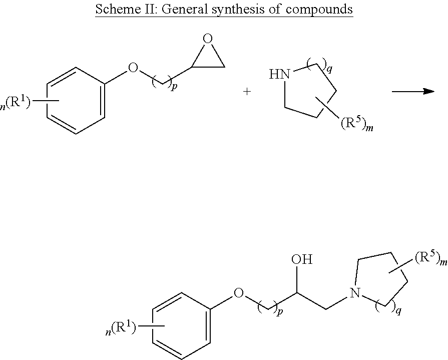

wherein:
n is 1, 2, or 3;
p is 1, 2, 3, 4, or 5;
m is 1, 2, or 3;
q is 1 or 2; and
each R¹ is independently halo, alkyl, or haloalkyl;
R⁵ is aryl, heteroaryl, alky, NH₂, NHR^{A}, or NR^{A}R;
R^{A} is C₁₋₆ alkyl, C₁₋₆ alkenyl, or C₁₋₆ alkynyl, each of which are optionally substituted with —OH, or C₁₋₆ alkoxy;
R^{B} is C₁₋₆ alkyl, C₁₋₆ alkenyl, or C₁₋₆ alkynyl, each of which are optionally substituted with —OH, or C₁₋₆ alkoxy.

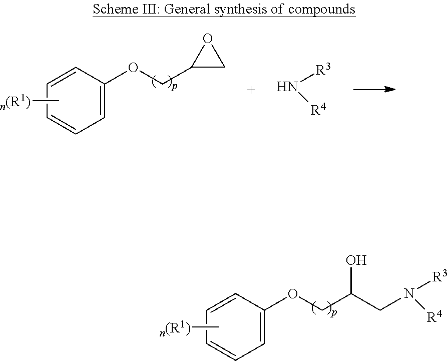

wherein:
n is 1, 2, or 3;
p is 1, 2, 3, 4, or 5;
q is 1 or 2; and
each R¹ is independently halo, alkyl, or haloalkyl;
R³ is —H, alkyl, alkenyl, or alkynyl;
R⁴ is alkyl, alkenyl, alkynyl, cycloalkyl, or heterocyclyl, each of which is optionally substituted with one or more R⁵;
or R³ and R⁴ are taken together to form a heterocyclyl, which is optionally substituted with one or more R⁵,
R⁵ is aryl, heteroaryl, alkyl, NH₂, NHR^{A}, or NR^{A}R^{B}, or alkyl substituted with —NH₂;
R^{A} is alkyl, alkenyl, alkynyl, each of which are optionally substituted with —OH or alkoxy; and
R^{B} is alkyl, alkenyl, or alkynyl.

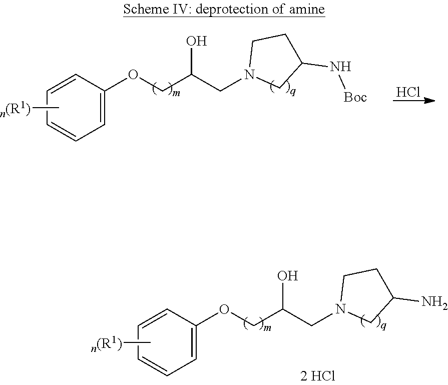

wherein:
n is 1, 2, or 3;
m is 1, 2, 3, 4, or 5;
q is 1 or 2; and
each R¹ is independently halo, alkyl, or haloalkyl.

### Methods of Treatment

In embodiments, the disclosure provides methods of treating a bacterial infection in a subject in need thereof, comprising administering to the subject a pharmaceutically acceptable amount one or more compounds of Formula (I), (II), (III), (IV), (V), or a stereoisomer or pharmaceutically acceptable salt thereof. In embodiments, the disclosure provides methods of inhibiting bacterial efflux pumps in a subject having a bacterial infection, comprising administering to the subject a pharmaceutically acceptable amount one or more compounds of Formula (I), (II), (III), (IV), (V), or a stereoisomer or pharmaceutically acceptable salt thereof.

In embodiments, the bacterial infection is caused by a Gram-negative bacterium. In embodiments, the Gram-negative bacterium may be an intracellular pathogen.

In embodiments, the bacterial infection is caused by one or more Salmonella sp., Acinetobacter sp., Actinobacillus sp., Aeromonas sp., Bacteroide sp., Bordetella sp., Brucella sp., Burkholderia sp., Prevotella sp., Porphyromonas sp., Campylobacter sp., Citrobacter sp., Edwarsiella sp., Eikenella sp., Enterobacter sp., Escherichia sp., Francisella sp., Haemophilus sp., Helicobacter sp., Kingella sp., Klebsiella sp., Legionella sp., Moraxella sp., Morganella sp., Neisseria sp., Pasteurella sp., Plesiomonas sp., Proteus sp., Providencia sp., Pseudomonas sp., Salmonella sp., Serratia sp., Shigella sp., Stenotrophomonas sp., Streptobacillus sp., Vibrio sp., Yersinia sp., Chlamydophila sp., Ricketsia sp., Coxiella sp., Ehrlichia sp., or Bartonella sp.

Acinetobacter baumannii, Acinetobacter haemolyticus, Actinobacillus actinomycetemcomitans, Aeromonas hydrophila, Bacteroides fragilis, Bacteroides theataioatamides theataioatamides theataioatides distasonis, Bacteroides ovatus, Bacteroides vulgatus, Bordetella pertussis, Brucella melitensis, Burkholderia cepacia, Burkholderia pseudomallei, Burkholderia mallei, Prevotella corporis, Prevotella intermedia, Prevotella endodontalis, Porphyromonas asaccharolytica, Campylobacter jejuni, Campylobacter coli, Campylobacter fetus, Citrobacter freundii, Citrobacter koseri, Edwarsiella tarda, Eikenella corrodens, Enterobacter cloacae, Enterobacter aerogenes, Enterobacter agglomerans, Escherichia coli, Francisella tularensis, Haemophilus influenzae, Haemophilus ducreyi, Helicobacter pylori, Kingella kingae, Klebsiella pella pneumonia ella rhinoscleromatis, Klebsiella ozaenae, Legionella penumophila, Moraxella catarrhalis, Morganella morganii, Neisseria gonorrhoeae, Neisseria meningitidis, Pasteurella multocida, Plesiomonas shigelloides, Proteus mirabilis, Proteus vulgaris, Proteus penneri, Proteus myxofaciens, Providencia stuartii, Providencia rettgeri, Providencia alcalifaciens, Pseudomonas aeruginosa, Pseudomonas fluorescens, Salmonella typhi, S. enterica, Salmonella paratyphi, Serratia marcescens, Shigella flexneri, Shigella boydii, Shigella sonnei, Shigella dysenteriae, Stenotrophomonas maltophilia, Streptobacillus moniliformis, Vibrio cholerae, Vibrio parahaemolyticus, Vibrio vulnificus, Vibrio alginolyticus, Yersinia enterocolitica, Yersinia pestis, Yersinia pseudotuberculosis, Chlamydophila pneumoniae, Chlamydophila trachomatis, Ricketsia prowazekii, Coxiella burnetii, Ehrlichia chaffeensis or Bartonella hensenae.

In embodiments, the bacterial infection is caused by one or more Salmonella sp., In embodiments, the Salmonella sp is S. enterica serovar Typhimurium. In embodiments, the bacterial infection is caused by E. coli. In embodiments, the bacterial infections is caused by Klebsiella pneumonia. In embodiments, the bacterial infections is caused by Enterobacter cloacae.

In embodiments, the compounds of the disclosure may be administered in combination (separately, simultaneously, e.g., as part of the same composition in a combination product, or sequentially) with one or more antibiotics. In embodiments, the antibiotic is a macrolide, tetracycline, fluoroquinolone, penicillin, cephalosporin, aminoglycoside, sulfonamide, beta-lactam, tetracycline, trimethoprim-sulfamethoxazole, chloramphenicol, or lincosamide.

In embodiments, the antibiotic is penicillin G, penicillin V, methicillin, oxacillin, cloxacillin, dicloxacillin, nafcillin, ampicillin, amoxicillin, carbenicillin, ticarcillin, mezlocillin, piperacillin, azlocillin, temocillin, cepalothin, cephapirin, cephradine, cephaloridine, cefazolin, cefamandole, cefuroxime, cephalexin, cefprozil, cefaclor, loracarbef, cefoxitin, cefmetazole, cefotaxime, ceftizoxime, ceftriaxone, cefoperazone, ceftazidime, cefixime, cefpodoxime, ceftibuten, cefdinir, cefpirome, cefepime, BAL5788, BAL9141, imipenem, ertapenem, meropenem, astreonam, clavulanate, sulbactam, tazobactam, streptomycin, neomycin, kanamycin, paromycin, gentamicin, tobramycin, amikacin, netilmicin, spectinomycin, sisomicin, dibekalin, isepamicin, tetracycline, chlortetracycline, demeclocycline, minocycline, oxytetracycline, methacycline, doxycycline, erythromycin, azithromycin, clarithromycin, telithromycin, ABT-773, lincomycin, clindamycin, vancomycin, oritavancin, dalbavancin, teicoplanin, quinupristin and dalfopristin, sulphanilamide, para-aminobenzoic acid, sulfadiazine, sulfisoxazole, sulfamethoxazole, sulfathalidine, linezolid, nalidixic acid, oxolinic acid, norfloxacin, perfloxacin, enoxacin, ofloxacin, ciprofloxacin, temafloxacin, lomefloxacin, fleroxacin, grepafloxacin, sparfloxacin, trovafloxacin, clinafloxacin, gatifloxacin, moxifloxacin, gemifloxacin, sitafloxacin, metronidazole, daptomycin, garenoxacin, ramoplanin, faropenem, polymyxin, tigecycline, AZD2563, or trimethoprim.

In embodiments, the compounds disclosed herein are used in the treatment of Gram-negative bacterial infections which have developed resistance to antibiotics. The language “resistance” and “antibacterial resistance” refers to bacteria that are able to survive exposure to one or more antibiotics. In embodiments, the bacteria is resistant to one or more of an aminoglycoside antibiotic (e.g., amikacin, gentamicin, kanamycin, neomycin, netilmicin, tobramycin, paromomycin, spectinomycin), an ansamycin antibiotic (e.g., rifaximin, streptomycin), a carbapenem antibiotic (e.g., ertapenem, doripenem, imipenem/cilastatin, meropenem), a cephalosoprin antibiotic (e.g., cefadroxil, cefaxolin, cefatolin, cefalexin, cefaclor, cefamandole, cefoxitin, cefprozil, cefuroxime, cefisime, cefdinir, cefditoren, cefoperazone, cefotaxime, cefpodoxime, ceftazidime, certibuten, ceftizoxime, ceftriaxone, cefepime, ceftarolin fosamil, ceftobiprole), a glycopeptide antibiotic (e.g., teicoplanin, vancomycin, telavancin), a lincosamide antibiotic (e.g., clindamycin, lincomycin), daptomycin, a macrolide antibiotic (e.g., azithromycin, clarithromycin, dirithromycin, erythromycin, roxithromycin, troleandomycin, telithromycin, spiramycin), aztreonam, furazolidone, nitrofuantoin, an oxazolidinone antibiotic (e.g., linezolid, posizolid, radezolid, torezolid), a penicillin antibiotic (e.g., amoxacillin, ampicillin, azlocillin, carbenicillin, cloxacillin, dicloxacillin, flucloxacillin, mezlocillin, methicillin, nafcillin, oxacillin, penicillin, piperacillin, temocillin, ticarcillin), amoxicillin/clavulante, ampicillin/sulbactam, piperacillin/tazobactam, ticarcillin/clavulanate, a quinolone antibacterial (e.g., ciprofloxacin, enoxacin, gatifloxacin, gemifloxacin, levofloxacin, lomefloxacin, moxifloxacin, nalidixic acid, norfloxacin, ofloxacin, trovafloxin, grepafloxacin, sparfloxacin, temafloxacin), a suflonamide antibiotic (e.g., mafenide, sulfacetamide, sulfadiazine, silver sulfadiazine, sulfadimethoxine, sulfamethizole, sulfamethoxazole, sulfanilimide, sulfasalazine, sulfisoxazole, trimethoprim/sulfamethoxazole—TMP-SMX) and a tetracycline antibiotic (e.g., demeclocycline, doxycycline, minocycline, oxytetracycline, tetracycline, tigeclycline).

In embodiments where the compounds of the disclosure are used in combination with an antibiotic, the compounds reduce the MIC of the antibiotic (e.g., as measured in Example 2) by about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, or about 10 fold, including all values and ranges therein. In embodiments where the compounds of the disclosure are used in combination with an antibiotic, the dose of the antibiotic may be reduced (compared to the dose of the antibiotic administered in the absence of the disclosed compounds) by about 10%, about 15%, about 20%, about 25%, about 30%, about 35%, about 40%, about 45%, about 50%, about 55%, about 60%, about 65%, about 70%, about 75%, about 80%, about 85%, about 90% or more, including all values and ranges therein. In embodiments, the compounds of the disclosure allow for an antibiotic to be effective at the same dose at which the antibiotic was ineffective when administered in the absence of one of the disclosed compounds.

In embodiments, the disclosure provides methods of increasing sensitivity of bacteria to an antibiotic treatment by administering the compounds of the disclosure and one or more antibiotics.

In embodiments, the method comprises administering a pharmaceutically acceptable amount of one or more compounds of Formula (I), (II), (III), (IV), (V), or a stereoisomer or pharmaceutically acceptable salt thereof and one or more antibiotics to a subject in need thereof. In embodiments, the method increases sensitivity of Gram-negative bacteria to an antibiotic. In embodiments, the compounds of the disclosure are capable of rendering an antibiotic resistant strain of bacteria sensitive to the antibiotic to which it is otherwise resistant. In embodiments, the antibiotic is a macrolide, tetracycline, fluoroquinolone, penicillin, cephalosporin, aminoglycoside, sulfonamide, beta-lactam, tetracycline, trimethoprim-sulfamethoxazole, chloramphenicol, or lincosamide.

In embodiments where the compounds of the disclosure are used in combination with an antibiotic, the compounds increase sensitivity of the antibiotic by reducing the MIC of the antibiotic by about 1.1 fold, about 1.2 fold, about 1.3 fold, about 1.4 fold, about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, or about 10 fold, including all values and ranges therein. In embodiments where the compounds of the disclosure are used in combination with an antibiotic, the compounds increase sensitivity of the antibiotics by reducing dose and/or IC₅₀ of the antibiotic (compared to the dose of the antibiotic administered in the absence of the disclosed compounds) by about 10%, about 15%, about 20%, about 25%, about 30%, about 35%, about 40%, about 45%, about 50%, about 55%, about 60%, about 65%, about 70%, about 75%, about 80%, about 85%, about 90% or more, including all values and ranges therein. In embodiments, the compounds of the disclosure allow for an antibiotic to be effective at the same dose at which the antibiotic was ineffective when administered in the absence of one of the disclosed compounds.

In embodiments, the disclosure also provides methods of reversing or decreasing antibiotic resistance of an antibiotic-resistant Gram-negative bacteria. In embodiments, the method comprises administering a pharmaceutically acceptable amount of one or more compounds of Formula (I), (II), (III), (IV), (V), or a stereoisomer or pharmaceutically acceptable salt thereof and one or more antibiotics to a subject in need thereof. In embodiments, the antibiotic is a macrolide, tetracycline, fluoroquinolone, penicillin, cephalosporin, aminoglycoside, sulfonamide, beta-lactam, tetracycline, trimethoprim-sulfamethoxazole, chloramphenicol, or lincosamide.

In embodiments, reversing or decreasing of antibiotic resistance is determined by assessing a reduction in growth rate of the antibiotic resistant strain in the presence of the compounds and one or more antibiotics. In embodiments, the compounds of the present disclosure administered in combination with antibiotics reverse or decrease antibiotic resistance by reducing bacterial pathogenicity, inhibiting or killing antibiotic-resistant bacteria, preventing biofilm formation, preventing septic shock, treating sepsis, and/or increasing bacterial susceptibility to antibiotics to which they previously exhibited resistance. In embodiments where the compounds of the disclosure are used in combination with an antibiotic, the antibiotic-resistant bacterial pathogenicity or growth rate may be reduced (compared to the dose of the antibiotic administered in the absence of the disclosed compounds) by about 10%, about 15%, about 20%, about 25%, about 30%, about 35%, about 40%, about 45%, about 50%, about 55%, about 60%, about 65%, about 70%, about 75%, about 80%, about 85%, about 90% or more, including all values and ranges therein. In embodiments where the compounds of the disclosure are used in combination with an antibiotic, the compounds reverses or decreases antibiotic resistance by reducing the MIC of the antibiotic by about 1.1 fold, about 1.2 fold, about 1.3 fold, about 1.4 fold, about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, or about 10 fold, including all values and ranges therein.

### Pharmaceutical Compositions

In some embodiments of the present disclosure, a pharmaceutical composition comprises a therapeutically effective amount of one or more compounds of Formula (I), (II), (III), (IV) or (V), or a stereoisomer or pharmaceutically acceptable salt thereof.

In embodiments, pharmaceutical compositions comprising one or more compounds disclosed herein, or a stereoisomer or pharmaceutically acceptable salt thereof, and a pharmaceutically acceptable excipient or adjuvant is provided. The pharmaceutically acceptable excipients and adjuvants are added to the composition or formulation for a variety of purposes. In embodiments, the pharmaceutical composition comprises a pharmaceutically acceptable carrier, binder, and/or diluent. In embodiments, the pharmaceutical composition may contain additional materials useful in physically formulating various dosage forms of the compositions of the present disclosure, such as dyes, flavoring agents, preservatives, antioxidants, opacifiers, thickening agents, stabilizers lubricants, wetting agents, emulsifiers, salts for influencing osmotic pressure, buffers, colorings, flavorings and/or aromatic substances.

In certain embodiments, the pharmaceutical compositions of the present disclosure may additionally contain other adjunct components conventionally found in pharmaceutical compositions, at their art-established usage levels. Thus, for example, the pharmaceutical compositions may contain additional, compatible, pharmaceutically-active materials such as antipruritics, astringents, local anesthetics or anti-inflammatory agents.

The compounds of the present disclosure may be formulated for administration by a variety of means including orally and parenterally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

The compounds disclosed herein can be formulated in accordance with the routine procedures adapted for desired administration route. Accordingly, the compounds disclosed herein can take such forms as suspensions, solutions or emulsions in oily or aqueous vehicles, and may contain suspending, stabilizing and/or dispersing agents. The compounds disclosed herein can also be formulated as a preparation for injection.

In certain embodiments, a pharmaceutical composition of the present disclosure is prepared using known techniques, including, but not limited to mixing, dissolving, granulating, dragee-making, levigating, emulsifying, encapsulating, entrapping or tableting processes.

In embodiments, the pharmaceutical composition may be a solid, powder, liquid and a gel. In embodiments, the pharmaceutical is a solid (e.g., a powder, tablet, a capsule, granulates, and/or aggregates). In certain of such embodiments, the solid pharmaceutical composition comprises one or more excipients known in the art, including, but not limited to, starches, sugars, diluents, granulating agents, lubricants, binders, and disintegrating agents

Solid carriers suitable for use in the present application include, but are not limited to, sugars and sugar alcohols (e.g., lactose, glucose, mannitol, and the like) starch, methyl-cellulose, magnesium stearate, dicalcium phosphate, calcium phosphate, magnesium stearate, talc, sugars, dextrin, starch, gelatin, cellulose, and polyvinylpyrrolidine. A solid carrier can further include one or more substances acting as flavoring agents, lubricants, solubilizers, suspending agents, fillers, glidants, compression aids, binders or tablet-disintegrating agents. A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropylmethyl cellulose), lubricant, inert diluent, preservative, and/or disintegrant (e.g., sodium starch glycolate, cross-linked povidone, cross-linked sodium carboxymethyl cellulose). Molded tablets may be made by molding in a suitable machine a mixture of the powdered compound moistened with an inert liquid diluent.

In embodiments, the pharmaceutical composition is formulated as a liquid. Liquid pharmaceutical composition suitable for use in the present disclosure include solutions, suspensions, emulsions, syrups, elixirs and pressurized compounds. The active ingredient can be dissolved or suspended in a pharmaceutically acceptable liquid carrier such as water, an organic solvent, a mixture of both or pharmaceutically acceptable oils or fats. In embodiments, the liquid solution may be an aqueous or non-aqueous solution.

Examples of non-aqueous carriers include, but are not limited to, propylene glycol, polyethylene glycol, vegetable oils such as olive oil, and injectable organic esters.

Aqueous carriers suitable for use in the present application include, but are not limited to, water, ethanol, alcoholic/aqueous solutions, glycerol, emulsions or suspensions, including saline and buffered media.

Parenteral carriers suitable for use in the present application include, but are not limited to, sodium chloride solution, Ringer's dextrose, dextrose and sodium chloride, lactated Ringer's and fixed oils. Intravenous carriers include fluid and nutrient replenishers, electrolyte replenishers such as those based on Ringer's dextrose and the like. Preservatives and other additives can also be present, such as, for example, antimicrobials, antioxidants, chelating agents, inert gases and the like. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators.

In embodiments, the amount of the compound disclosed herein, or a stereoisomer or pharmaceutically acceptable salt thereof, can be administered at about 0.001 mg/kg to about 100 mg/kg body weight (e.g., about 0.01 mg/kg to about 10 mg/kg or about 0.1 mg/kg to about 5 mg/kg). In embodiments, the amount of the compound disclosed herein, or a stereoisomer or pharmaceutically acceptable salt thereof, can be administered at about 0.1 mg to about 1,000 mg (e.g., about 0.1 mg to about 500 mg/kg or about 0.1 mg/kg to about 100 mg/kg).

### EXAMPLES

The following experiments were performed using the SAFIRE assay described in U.S. Publication 2020/0022961, which is incorporated by reference in its entirety for all purposes. SAFIRE (Screen for Anti-infectives using Fluorescence microscopy of IntracellulaR Enterobacteriaceae) uses cell culture as a surrogate to identify compounds that prevent intracellular replication of a model Gram-negative bacterial human pathogen. The method identifies compounds that prevent bacterial growth in broth culture and compounds that decrease the load of Gram-negative bacteria inside of macrophages at concentrations≤10 μM but have no effect on bacterial growth in standard Mueller Hinton broth (MHB) at 100 μM. Thus, SAFIRE enables the discovery of compounds that are effective against Gram-negative bacteria in host cells and in whole animals, whether or not they are antibacterial in broth.

### Example 1. Inhibition Curves

### Bacterial Strains

The wild-type S. enterica serovar Typhimurium strain, SL1344, was initially isolated from the blood of an infected calf. For screening and validation in macrophages, SL1344 sifB::gfp was grown in Luria-Bertani Broth (LB) with 30 μg/ml streptomycin and 30 μg/ml kanamycin to saturation overnight, diluted to an OD of 0.001 and frozen in 100 μL aliquots in 20% glycerol at −80° C. Prior to infection, aliquots were grown in 5 mL cultures of LB with 30 μg/ml streptomycin and 30 μg/ml kanamycin for 18 hours at 37° C. with aeration. Bacterial strains were routinely grown in LB with antibiotics: 30 μg/ml streptomycin, 30 μg/ml kanamycin, 50 μg/ml ampicillin, 10 μg/ml tetracycline, and/or 1.15 μg/ml meropenem. The acrAB::kan and macAB::kan strains were constructed according to known methods. S. enterica subsp. enterica, serovar Typhimurium strain S10801, NR-22067 is a multidrug resistant isolate from a calf with sepsis. This strain and others as indicated were obtained through BEI resources, NIAID, NIH.

### Cell Culture

Murine macrophage-like RAW 264.7 cells and HeLa human epithelial cells were obtained from the American Type Tissue Collection. BMDMs were isolated as previously described. Briefly, marrow was flushed from the femurs of 1- to 4-month-old 129SvEvTac mice (Taconic Laboratories) bred in-house. Mononuclear cells were separated using Histopaque-1083 (Sigma), washed, and directly seeded into assay plates at 1×10₅ cells/ml in complete medium supplemented with 35% conditioned media from 3T3 cells expressing MCSF. Media were refreshed three days later. After 1 week, media were replaced with 100 μL fresh media and cells were infected as described below. All three types of cells were grown in DMEM high glucose (Sigma) supplemented with 10% fetal bovine serum, 2 mM L-glutamine, 1 mM sodium pyruvate, 10 mM HEPES, and 50 μMβ-mercaptoethanol. Cells were maintained in a 5% CO₂ humidified atmosphere at 37° C. For screening, frozen aliquots of RAW 264.7 were thawed and allowed to expand for three days prior to seeding; other experiments were performed with cultures between passages four and 20.

### Bacterial Infections for SAFIRE and CFU Plating

SAFIRE—RAW264.7 macrophages (7×10₃ in 40 μL or 5×10₄ in 100 μL) were seeded, respectively, in 384- or 96-well black-walled glass-bottomed plates (Brooks Automation). Twenty four hours post-seeding, bacteria in 20 or 50 μL PBS were added to a final concentration of 1×10₇ CFU/mL, conditions yielding infection of approximately 70% of macrophages at 18 hours post-infection with minimal macrophage toxicity. The sifB::gfp bacterial reporter strain was used to minimize green signal from extracellular bacteria. Forty-five minutes after bacterial addition, 20 or 50 μL gentamicin was added to a final concentration of 40 μg/mL, which did not affect intracellular infection but inhibited replication of extracellular bacteria. At two hours post-infection, 200 or 500 nL compound was added using a pin tool (CyBio) to yield a final concentration of 25 μM. Each assay plate included rifampicin and DMSO controls. In some experiments, media were removed and replaced with fresh media containing 40 μg/mL gentamicin and increasing concentrations of the compounds of the disclosure. At 17.5 hours post-infection, PBS containing MitoTracker Red CMXRos (Life Technologies) was added to a final concentration of 300 nM or 100 nM, for 384- or 96-well, respectively. Thirty minutes later, 16% paraformaldehyde was added to a final concentration of 1±2% and incubated at room temperature for 15 minutes. Wells were washed twice with PBS and stained for 20 minutes with 1 μMDAPI; wells were washed twice and stored in 90% glycerol in PBS until imaging. The Z′-factor of the screening platform was 0.59 and 0.48 in 96-well and 384-well plates, respectively, within published ranges for complicated cell-based screens.

Infections of HeLa cells with S. Typhimurium were performed as above except that 1×10₄ cells were seeded, and cells were infected with S. Typhimurium constitutively expressing GFP from the rpsM locus because sifB::gfp is poorly expressed in Hela cells. In addition, plates were spun for five minutes at 500× g after addition of bacteria to enhance infection.

CFU—Infections were performed as described above, except cells were seeded in 96-well tissue culture coated plates (Greiner). At 18 hours post-infection, wells were washed three times in PBS, lysed with 30 μL 0.1% Triton X-100, diluted and plated to determine CFU.

IC50 values for the compounds of the disclosure were measured using SAFIRE across at least eight concentrations using 2-fold dilutions ranging from 50 to 0.001 UM and are provided in Table A below.

In addition, toxicity was measured according the following procedure: 1) analyzing macrophage cell morphology; and 2) using MATLAB to count cells (cells that lift up and float after treatment are not within the appropriate Z plane to be counted, and thus are considered dead). If 70% of the cells are adherent (as measured by comparing the number of cells before and after treatment), the compound is considered not toxic. Compounds that pass #1 and #2 above are considered to have a toxicity “>50 μM”. Compounds that do not pass either #1 or #2 are considered to have a toxicity “<25 μM”. The data is shown below in Table A.

TABLE A

| Compound | | IC50 | Toxicity |
|---|---|---|---|
| no. | Structure | (μM) | (μM) |
| 1 | 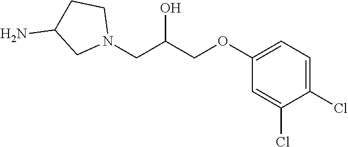 [SMILES: NC1CCN(CC(O)COc2ccc(Cl)c(Cl)c2)C1] | <1 | >50 |
| 2 | 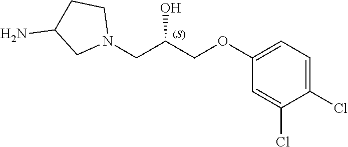 | <1 | >50 |
| 3 | 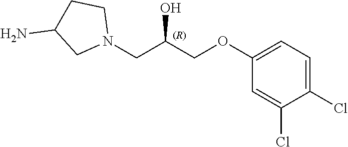 | <1 | >50 |
| 4 | 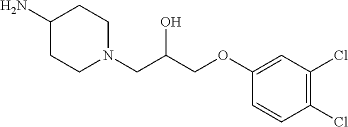 [SMILES: NC1CCN(CC(O)COc2ccc(Cl)c(Cl)c2)CC1] | <1 | <25 |
| 5 | 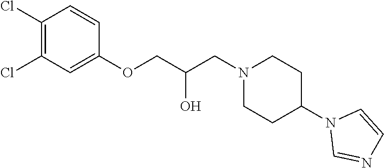 [SMILES: OC(COc1ccc(Cl)c(Cl)c1)CN1CCC(n2ccnc2)CC1] | <1 | >50 |
| 6 | 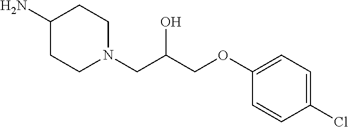 [SMILES: NC1CCN(CC(O)COc2ccc(Cl)cc2)CC1] | <1 | >50 |
| 7 | 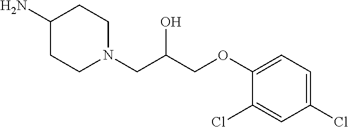 [SMILES: NC1CCN(CC(O)COc2ccc(Cl)cc2Cl)CC1] | <1 | <50 but >25 |
| 8 | 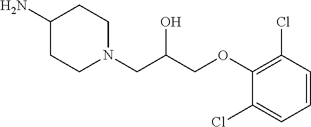 [SMILES: NC1CCN(CC(O)COc2c(Cl)cccc2Cl)CC1] | <1 | >50 |
| 9 | 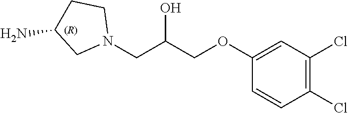 | <1 | >50 |
| 10 | 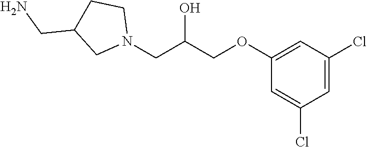 [SMILES: NCC1CCN(CC(O)COc2cc(Cl)cc(Cl)c2)C1] | <1 | <50 but >25 |
| 11 | 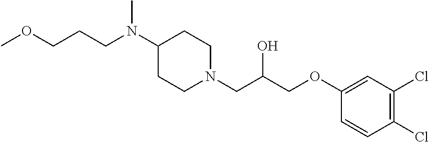 [SMILES: COCCCN(C)C1CCN(CC(O)COc2ccc(Cl)c(Cl)c2)CC1] | <1 | >50 |
| 12 | 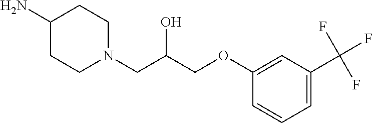 [SMILES: NC1CCN(CC(O)COc2cccc(C(F)(F)F)c2)CC1] | <1 | >50 |
| 13 | 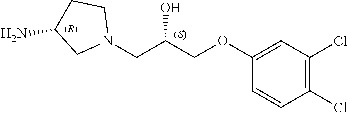 | <1 | >50 |
| 14 | 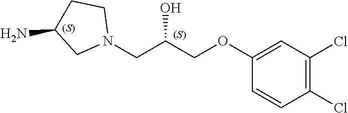 | >1 | >50 |
| 15 | 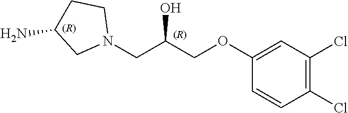 | <1 | >50 |
| 16 | 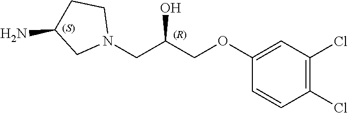 | <1 | >50 |
| 17 | 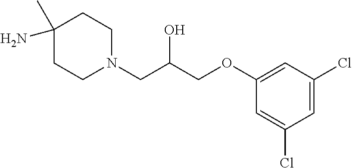 [SMILES: CC1(N)CCN(CC(O)COc2cc(Cl)cc(Cl)c2)CC1] | <1 | <25 |
| 18 | 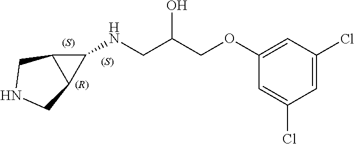 | <1 | >50 |
| 19 | 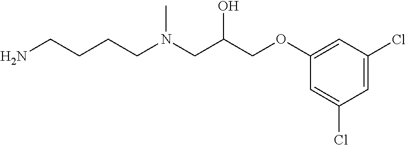 [SMILES: CN(CCCCN)CC(O)COc1cc(Cl)cc(Cl)c1] | <1 | <50 but >25 |

### Example 2. Spectrum of Broth Activity

To evaluate the ability of the compounds of the disclosure to sensitive bacteria to antibiotics, the compounds of the disclosure were combined with known antibiotics doxycycline, ciprofloxacin and chloramphenicol in several bacteria cell lines, including carbapenem resistant Enterobacteriaceae, and the minimum inhibitor concentration (MIC) was measured. MIC for the antibiotic alone (i.e., in the absence of the disclosed compounds) is provided in the first row labeled “None”. Comparator compounds, EPI 35 and PaβN, which have been reported to inhibit bacterial efflux pumps, and the compounds of the disclosure were added at 50 μg/mL, and the MIC of the antibiotic was revaluated in each cell line. These results are presented in Tables B-D. The compounds of the disclosure reduced the MIC of these antibiotic when used at concentrations of 50 μg/mL. At this concentration (50 μg/mL), the compounds of the disclosure lacked intrinsic antibacterial activity but a significant improvement in efficacy was still observed. Thus, the compounds of the disclosure may be used to potentiate the activity of antibiotics that have lost their efficacy due to efflux.

TABLE B

| | Doxycycline MIC (μg/mL) | | | | |
|---|---|---|---|---|---|
| Selected | S. enterica sp. | | | | |
| Compound | Enterica | E. coli | K. pneumoniae | K. pneumoniae | E. cloacae |
| 50 μg/mL in MHB | S10801 | BAA-2340 | BAA-2342 | BAA-1705 | BAA-2341 |
| None | 32 | 8 | 8 | 4 | 8 |
| EPI 35 | 8 | 8 | 8 | 8 | 8 |
| 1 | 8 | ≤1 | 2 | ≤1 | ≤1 |
| 3 | 8 | ≤1 | 2 | ≤1 | ≤1 |
| 4 | 8 | ≤1 | ≤1 | ≤1 | ≤1 |
| 7 | 8 | ≤1 | ≤1 | ≤1 | ≤1 |
| 10 | 8 | ≤1 | ≤1 | ≤1 | ≤1 |
| 16 | 8 | ≤1 | 2 | ≤1 | ≤1 |
| 17 | 8 | 2 | 4 | ≤1 | 2 |
| 18 | 4 | ≤1 | ≤1 | ≤1 | ≤1 |
| 19 | 8 | ≤1 | ≤1 | ≤1 | ≤1 |
| PAβN (25 μg/mL) | 8 | ≤1 | ≤1 | ≤1 | ≤1 |

TABLE C

| | PMB MIC | | | | |
|---|---|---|---|---|---|
| Selected | (μg/mL) | Ciprofloxacin MIC (μg/mL) | | | |
| Compound | E. Cloacae | E. coli | K. pneumoniae | K. pneumoniae | E. cloacae |
| 50 μg/mL in MHB | MB315 | BAA-2340 | BAA-2342 | BAA-1705 | BAA-2341 |
| None | 32 | 32 | 32 | >64 | 32 |
| EPI 35 | 1 | 32 | 32 | 64 | 32 |
| 1 | 32 | 8 | 8 | 32 | 16 |
| 3 | 32 | 16 | 16 | 32 | 16 |
| 4 | 16 | 8 | 8 | 16 | 8 |
| 7 | 32 | 8 | 8 | 32 | 16 |
| 10 | 32 | 16 | 16 | 32 | 16 |
| 16 | 32 | 16 | 16 | 32 | 16 |
| 17 | 16 | 16 | 16 | 32 | 16 |
| 18 | 1 | 8 | 8 | 16 | 8 |
| 19 | 32 | 32 | 32 | 64 | 16 |
| PAβN (25 μg/mL) | 32 | 16 | 16 | 64 | 16 |

TABLE D

| Selected | | | | | | |
|---|---|---|---|---|---|---|
| Compound | Chloramphenicol MIC (μg/mL) | | | | | |
| 50 μg/mL | E. coli | K. pneumoniae | E. cloacae | A. baumannii | A. baumannii | P. aeruginosa |
| in MHB | BAA-2340 | BAA-2342 | BAA-2341 | MB306 | MB309 | Focus MB0477 |
| None | 64 | 64 | 16 | >64 | >64 | 64 |
| EPI 35 | 64 | 64 | 16 | 64 | 64 | >64 |
| 1 | 16 | 16 | 2 | 64 | 64 | 64 |
| 3 | 16 | 16 | 2 | 64 | 64 | 64 |
| 4 | 8 | 8 | 2 | 64 | 64 | 64 |
| 7 | 8 | 8 | 2 | 32 | 64 | 64 |
| 10 | 8 | 8 | 2 | 32 | 64 | 4 |
| 16 | 16 | 16 | 2 | 32 | 64 | 64 |
| 17 | 16 | 16 | 4 | 32 | 64 | 64 |
| 18 | 8 | 8 | 2 | 32 | 32 | >64 |
| 19 | 16 | 16 | 2 | 64 | 64 | 16 |
| PAβN (25 | 8 | 8 | 2 | 32 | 64 | 1 |
| μg/mL) | | | | | | |

### Example 3. Methods of Making Compounds

The compounds disclosed herein can be prepared by general synthesis Schemes I-IV as shown below and/or by any other suitable methods. The compounds may be characterized according to any suitable method known in the art, such as NMR, UV, HPLC, LC-MS and TLC.

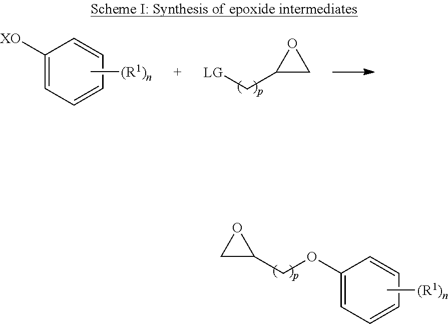

wherein:
X is H or a cation;
LG is a leaving group, such as a halide;
n is 1, 2, or 3;
p is 1, 2, 3, 4, or 5; and
each R¹ is independently halo, alkyl, haloalkyl.

The stirring mixture of 1.0 g of 3,4-fluorophenol and 1.5 equivalents of epichlorohydrin, 2.0 equivalents of Cs₂CO₃ was heated to 80° C. overnight. The reaction was quenched with water and the precipitate was purified by normal phase column chromatography using ethyl acetate and hexanes with good yield. The product is used in the next step. The product is characterized by LC-MS. Desired the mass was observed.

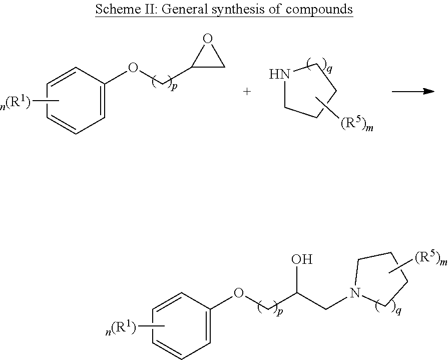

wherein:
n is 1, 2, or 3;
p is 1, 2, 3, 4, or 5;
m is 1, 2, or 3;
q is 1 or 2; and
each R¹ is independently halo, alkyl, or haloalkyl;
R⁵ is aryl, heteroaryl, alky, NH₂, NHR^{A}, or NR^{A}R^{B};
R^{A} is ₋₆ alkyl, C₁₋₆ alkenyl, or C₁₄ alkynyl, each of which are optionally substituted with —OH, or C₁₋₆ alkoxy; and
R^{B} is C₁₋₆ alkyl, C₁₋₆ alkenyl, or C₁₄ alkynyl, each of which are optionally substituted with —OH, or C₁₋₆ alkoxy.

The two starting materials (0.16 mMol) were mixed in 1:1 molar ratio in 1 mL of DI water and heated to 140° C. by microwave for 10 minutes. The resulting product is purified by reverse phase column chromatography to yield product. The product is characterized by LC-MS. Desired the mass was observed, as shown in TABLE E.

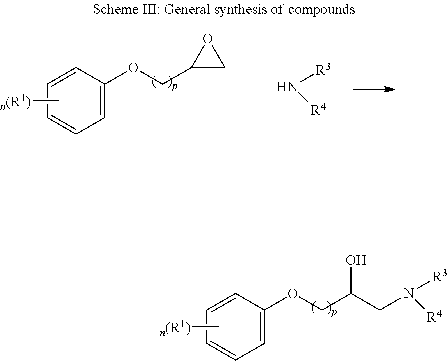

wherein:
n is 1, 2, or 3;
p is 1, 2, 3, 4, or 5;
q is 1 or 2; and
each R¹ is independently halo, alkyl, or haloalkyl;
R³ is —H, alkyl, alkenyl, or alkynyl;
R⁴ is alkyl, alkenyl, alkynyl, cycloalkyl, or heterocyclyl, each of which is optionally substituted with one or more R⁵;
or R³ and R⁴ are taken together to form a heterocyclyl, which is optionally substituted with one or more R⁵,
R⁵ is aryl, heteroaryl, alkyl, NH₂, NHR^{A}, or NR^{A}R^{B}, or alkyl substituted with —NH₂;
R^{A} is alkyl, alkenyl, alkynyl, each of which is optionally substituted with —OH or alkoxy; and
R^{B} is alkyl, alkenyl, or alkynyl.

The two starting materials (0.16 mMol) were mixed in 1:1 molar ratio in 1 mL of DI water and heated to 140° C. by microwave for 10 minutes. The resulting product is purified by reverse phase column chromatography to yield product. The product is characterized by LC-MS. Desired the mass was observed, as shown in TABLE E.

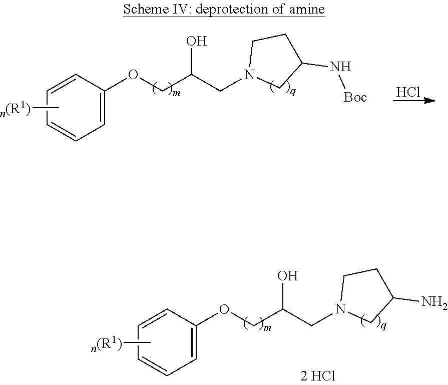

wherein:
n is 1, 2, or 3;
m is 1, 2, 3, 4, or 5;
q is 1 or 2; and
each R¹ is independently halo, alkyl, or haloalkyl.

The starting material (1.11 mMol) was dissolved in 3 ml 4N HCl in dioxane and stirred for overnight. The solvent was removed by evaporation and the final product was precipitated from diethyl ether, filtered, and dried to obtain the product as di-HCl salt. The product is characterized by LC-MS. Desired the mass was observed, as shown in TABLE E.

TABLE E
Measured mass and synthesis methods for compounds

| | | MW | Measured | Synthesis |
|---|---|---|---|---|
| No. | Structure | (g/mol) | (g/mol) | Schemes |
| 1 | 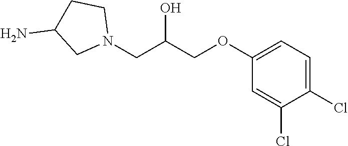 [SMILES: NC1CCN(CC(O)COc2ccc(Cl)c(Cl)c2)C1] | 293 | 294 | I, II, IV |
| 2 | 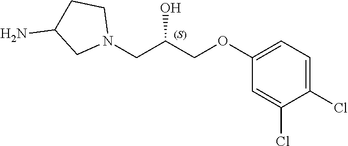 | 293 | 294 | I, II, IV |
| 3 | 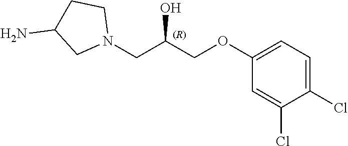 | 334 | 335 | I, II |
| 4 | 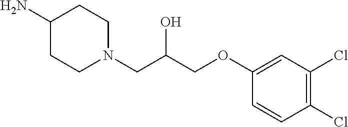 [SMILES: NC1CCN(CC(O)COc2ccc(Cl)c(Cl)c2)CC1] | 334 | 335 | I, II |
| 5 | 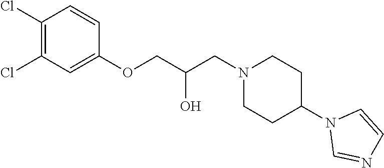 [SMILES: OC(COc1ccc(Cl)c(Cl)c1)CN1CCC(n2ccnc2)CC1] | 336 | 337 | I, II |
| 6 | 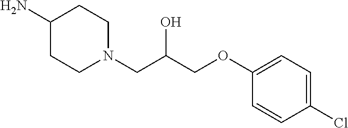 [SMILES: NC1CCN(CC(O)COc2ccc(Cl)cc2)CC1] | 336 | 337 | I, II |
| 7 | 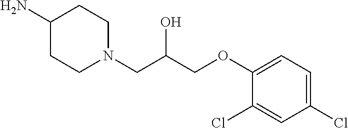 [SMILES: NC1CCN(CC(O)COc2ccc(Cl)cc2Cl)CC1] | 336 | 337 | I, II |
| 8 | 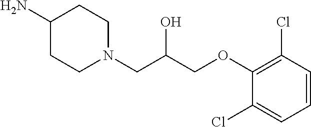 [SMILES: NC1CCN(CC(O)COc2c(Cl)cccc2Cl)CC1] | 336 | 337 | I, II |
| 9 | 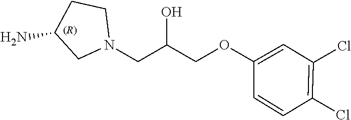 | 333 | 334 | I, II, IV |
| 10 | 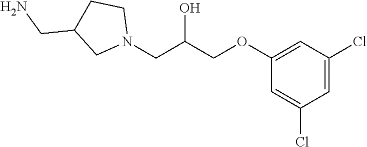 [SMILES: NCC1CCN(CC(O)COc2cc(Cl)cc(Cl)c2)C1] | 377 | 378 | I, II |
| 11 | 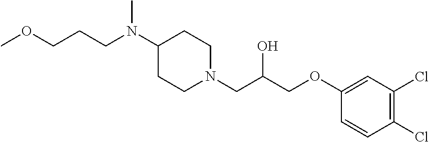 [SMILES: COCCCN(C)C1CCN(CC(O)COc2ccc(Cl)c(Cl)c2)CC1] | 350 | 351 | I, II |
| 12 | 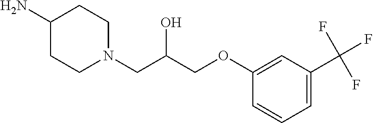 [SMILES: NC1CCN(CC(O)COc2cccc(C(F)(F)F)c2)CC1] | 394 | 395 | I, II |
| 13 | 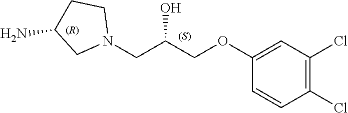 | 394 | 395 | I, II |
| 14 | 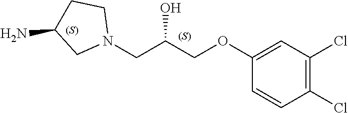 | 394 | 395 | I, II |
| 15 | 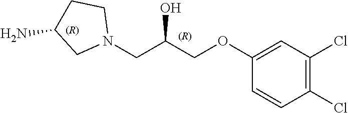 | 336 | 337 | I, II |
| 16 | 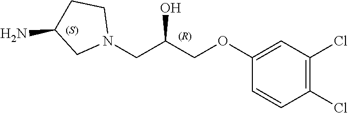 | 334 | 335 | I, II |
| 17 | 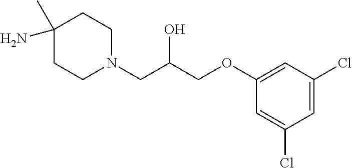 [SMILES: CC1(N)CCN(CC(O)COc2cc(Cl)cc(Cl)c2)CC1] | 356 | 357 | I, II |
| 18 | 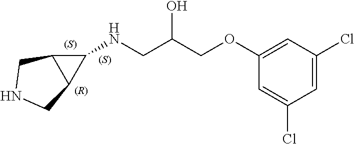 | 331 | 332 | I, III, IV |
| 19 | 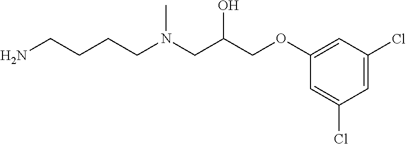 [SMILES: CN(CCCCN)CC(O)COc1cc(Cl)cc(Cl)c1] | 319 | 320 | I, III, IV |

1. A compound of Formula (I) 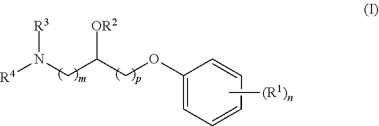
or a stereoisomer or pharmaceutically acceptable salt thereof,
wherein:
n is 1, 2, or 3;
p is 1, 2, 3, 4, or 5;
m is 1, 2, 3, 4, or 5;
each R¹ is independently halo, alkyl, or haloalkyl;
R² is —H, alkyl, alkenyl, or alkynyl;
R³ is —H, alkyl, alkenyl, or alkynyl;
R⁴ is alkyl, alkenyl, alkynyl, cycloalkyl, or heterocyclyl, each of which is optionally substituted with one or more R⁵;
or R³ and R⁴ are taken together to form a heterocyclyl, which is optionally substituted with one or more R⁵,
R⁵ is aryl, heteroaryl, alkyl, NH₂, NHR^{A}, or NR^{A}R^{B}, or alkyl-NH₂;
R^{A} is alkyl, alkenyl, or alkynyl, each of which is optionally substituted with —OH or alkoxy; and
R^{B} is alkyl, alkenyl, or alkynyl.

2. The compound of claim 1, wherein R³ and R⁴ are taken together to form a heterocyclyl, which is optionally substituted with one or more R⁵.

3. A compound of claim 1 or 2, having a structure of Formula (II): 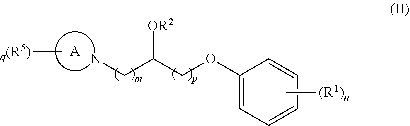
or a stereoisomer or pharmaceutically acceptable salt thereof,
wherein:
n is 1, 2, or 3;
p is 1, 2, 3, 4, or 5;
m is 1, 2, 3, 4, or 5;
q is 1, 2, or 3;
each R¹ is independently halo, alkyl, or haloalkyl;
R² is hydrogen, C₁₋₆ alkyl, C₁₋₆ alkenyl, or C₁₋₆ alkynyl;
A ring is heterocyclyl;
R⁵ is aryl, heteroaryl, alky, NH₂, NHR^{A}, or NR^{A}R^{B};
R^{A} is C₁₋₆ alkyl, C₁₋₆ alkenyl, or C₁₄ alkynyl, each of which is optionally substituted with —OH, or C₁₄ alkoxy; and
R^{B} is C₁₋₆ alkyl, C₁₋₆ alkenyl, or C₁₋₆ alkynyl, each of which is optionally substituted with —OH, or C₁₋₆ alkoxy.

4. The compound of claim 3, wherein:
the A ring is a 5-8 membered heterocyclyl, optionally having 1, 2, or 3 heteroatoms selected from N, O, or S in addition to the ring N shown in Formula (II).

5. The compound of claim 3 or 4, wherein the A ring is a 5-6 membered heterocyclyl.

6. The compound of any one of claims 3-5, wherein:
R⁵ is heteroaryl, NH₂, NHR^{A}, or NR^{A}R^{B};
R^{A} is C₁₋₆ alkyl optionally substituted with C₁₋₆ alkoxy; and
R^{B} is C₁₋₆ alkyl.

7. The compound of any one of claims 3-6, wherein:
p is 1 or 2.

8. The compound of any one of claims 3-7, wherein:
m is 1 or 2.

9. The compound of any one of claims 3-8, wherein:
q is 1.

10. The compound of claim 1, having a structure of Formula (III): 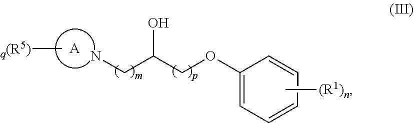
or a stereoisomer or pharmaceutically acceptable salt thereof,
wherein:
n is 1 or 2;
p is 1, 2, or 3;
m is 1, 2, or 3;
q is 1;
each R¹ is independently halo or haloalkyl;
A ring is a 5 or 6 membered heterocyclyl;
R⁵ is heteroaryl or C₁₋₆ alkyl, substituted with —NH₂, —NHR^{A}, or —NR^{A}R^{B};
R^{A} is C₁₋₆ alkyl which is optionally substituted with C₁₋₆ alkoxy; and
R^{B} is C₁₋₆ alkyl.

11. The compound of any one of claims 1-10, wherein each R¹ is Cl.

12. The compound of any one of claims 1-11, having a structure of Formula (IV): 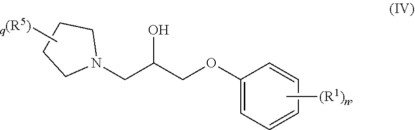
or a stereoisomer or pharmaceutically acceptable salt thereof,
wherein:
n is 1 or 2;
q is 1;
each R¹ is Cl or fluoroalkyl;
R⁵ is alkyl, —NH₂, or —NH₂, —NHR^{A}, or NR^{A}R^{B};
R^{A} is C₁₋₆ alkyl which are optionally substituted with C₁₋₆ alkoxy;
R^{B} is C₁₋₆ alkyl.

13. The compound of claim 12, wherein R⁵ alkyl-NH₂, or —NH₂.

14. The compound of claim 12 or 13, having the following structure: 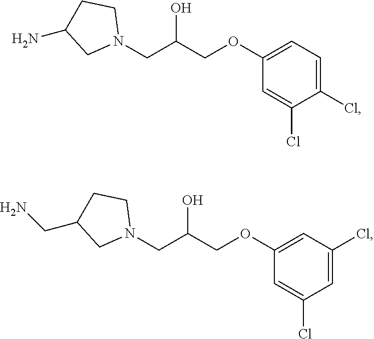 [SMILES: NC1CCN(CC(O)COc2ccc(Cl)c(Cl)c2)C1 ; NCC1CCN(CC(O)COc2cc(Cl)cc(Cl)c2)C1]
or a stereoisomer or pharmaceutically acceptable salt thereof.

15. The compound of claim 14, having the following structure: 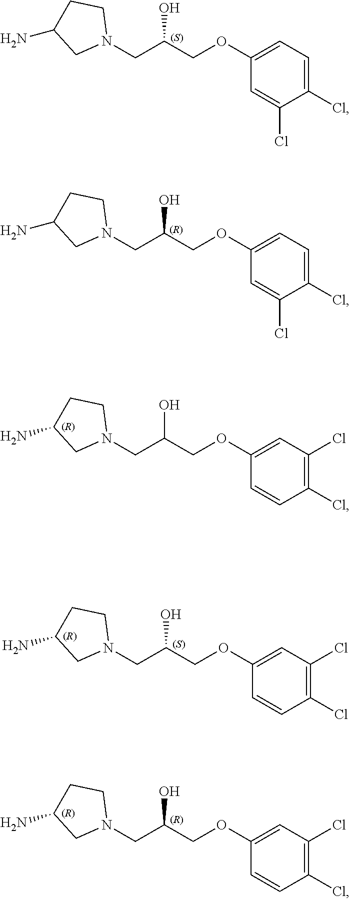
or a pharmaceutically acceptable salt thereof.

16. The compound of any one of claims 1-11, having a structure of Formula (V): 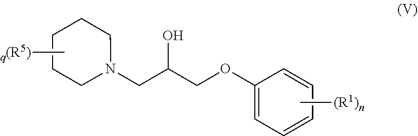
or a stereoisomer or pharmaceutically acceptable salt thereof,
wherein:
n is 1 or 2;
q is 1 or 2;
each R¹ is Cl or fluoroalkyl;
R⁵ is C₁₋₆ alkyl, heteroaryl, NH₂, NHR^{A}, or NR^{A}R^{B};
R^{A} is C₁₋₆ alkyl optionally substituted with C₁₋₆ alkoxy;
R^{B} is C₁₋₆ alkyl.

17. The compound of claim 16, wherein R⁵ is a 5-7 membered heteroaryl having 1, 2, or 3 heteroatoms selected from N or S.

18. The compound of claim 16 or 17, wherein R⁵ is a 5 membered heteroaryl having 1 or 2 N heteroatoms.

19. The compound of any one of claims 16-17, wherein R⁵ is imidazolyl.

20. The compound of any one of claims 16-19, having the following structure: 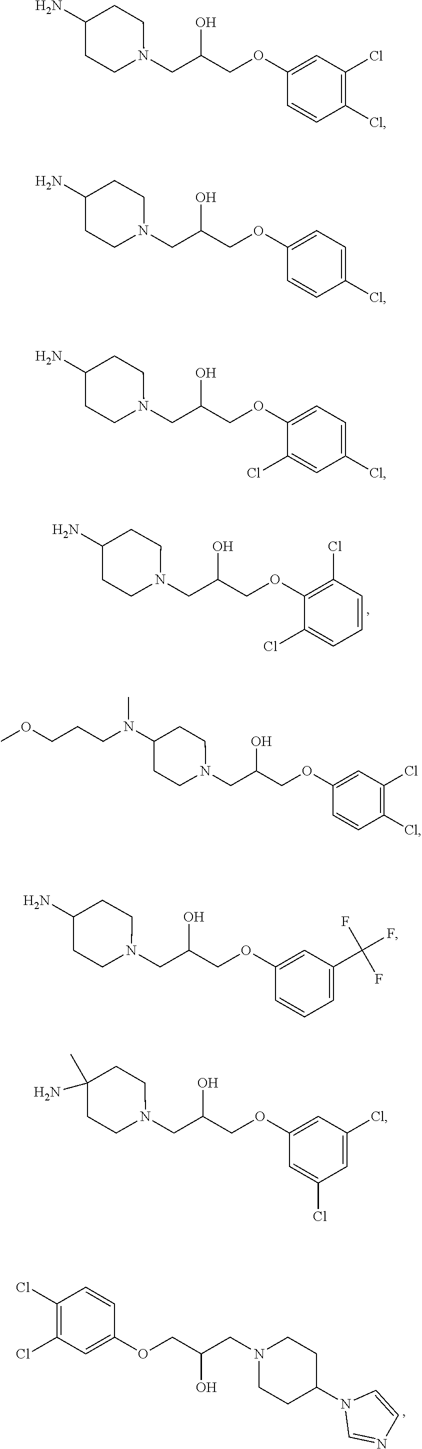
or a pharmaceutically acceptable salt thereof.

21. The compound of claim 1, wherein:
R³ is —H or alkyl.

22. The compound of claim 1 or 21, wherein
R⁴ is alkyl substituted with R⁵; and
R⁵ is NH₂.

23. The compound of claim 1 or 21, wherein:
R⁴ is heterocyclyl.

24. The compound of claim of 23, wherein:
the R⁴ is 5, 3 fused heterocyclyl.

25. The compound of any one of claims 21-24, wherein:
R² is H.

26. The compound of any one of claims 21-25, wherein:
n is 1 or 2, and R¹ is halo.

27. The compound of any one of claims 21-26, wherein:
m is 1 or 2.

28. The compound of any one of claims 21-27, wherein:
p is 1 or 2.

29. The compound of any one of claims 21-28, having one of the following structures: 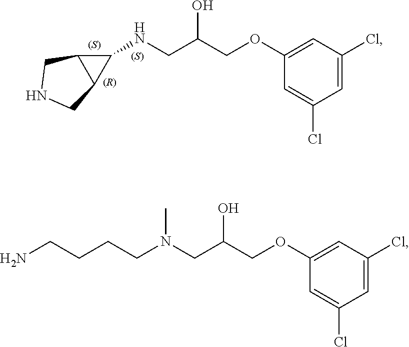
or a pharmaceutically acceptable salt thereof.

30. The compound of claim 1 having one of the following structures: 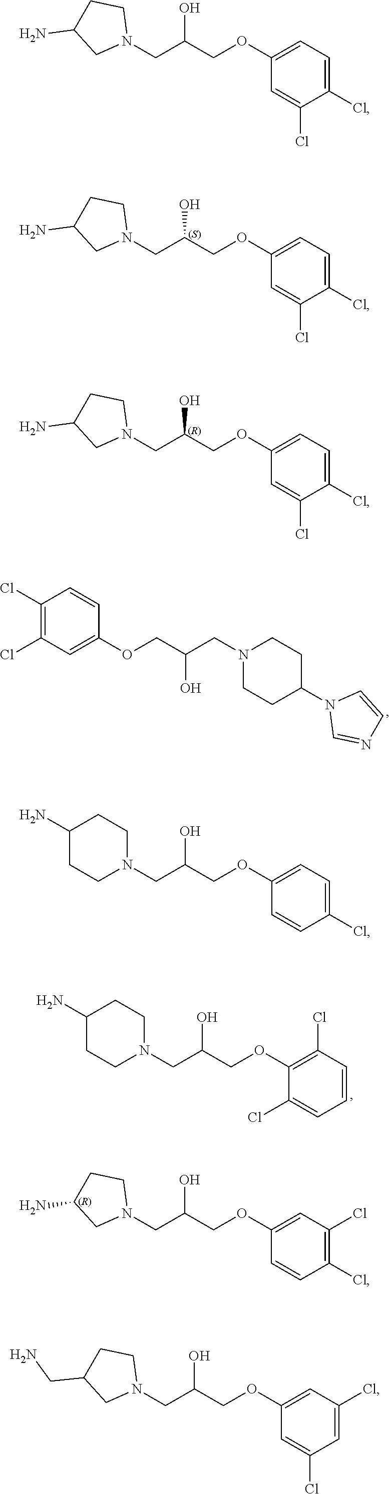 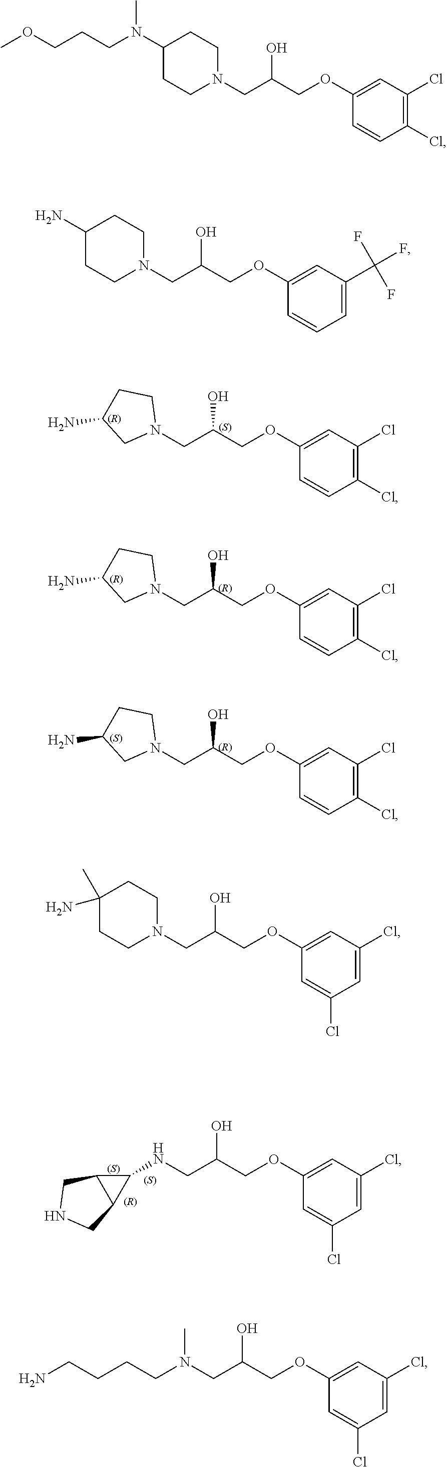
or a pharmaceutically acceptable salt thereof.

31. A pharmaceutical composition comprising therapeutically effective amount of a compound of any one of claims 1-30, and one or more pharmaceutically acceptable excipients.

32. A method of treating a bacterial infection in a subject in need thereof, comprising administering to the subject a pharmaceutically acceptable amount of a compound of any one of claims 1-30.

33. The method of claim 32, wherein the bacterial infection is caused by an intracellular pathogen.

34. The method of claim 32, wherein the bacterial infection is caused by Gram-negative bacteria.

35. The method of claim 34, wherein the bacterial infection is caused by one or more Salmonella sp., Acinetobacter sp., Actinobacillus sp., Aeromonas sp., Bacteroide sp., Bordetella sp., Brucella sp., Burkholderia sp., Prevotella sp., Porphyromonas sp., Campylobacter sp., Citrobacter sp., Edwarsiella sp., Eikenella sp., Enterobacter sp., Escherichia sp., Francisella sp., Haemophilus sp., Helicobacter sp., Kingella sp., Klebsiella sp., Legionella sp., Moraxella sp., Morganella sp., Neisseria sp., Pasteurella sp., Plesiomonas sp., Proteus sp., Providencia sp., Pseudomonas sp., Salmonella sp., Serratia sp., Shigella sp., Stenotrophomonas sp., Streptobacillus sp., Vibrio sp., Yersinia sp., Chlamydophila sp., Ricketsia sp., Coxiella sp., Ehrlichia sp., or Bartonella sp.

36. The method of 35, wherein the bacterial infection is caused by one or more Salmonella species.

37. The method of claim 36, wherein the Salmonella sp. is S. enterica serovar Typhimurium.

38. The method of claim 32, wherein the bacterial infection is caused by E. coli, Klebsiella pneumonia, or Enterobacter cloacae.

39. The method of any one of claims 32-38, wherein the compound is: 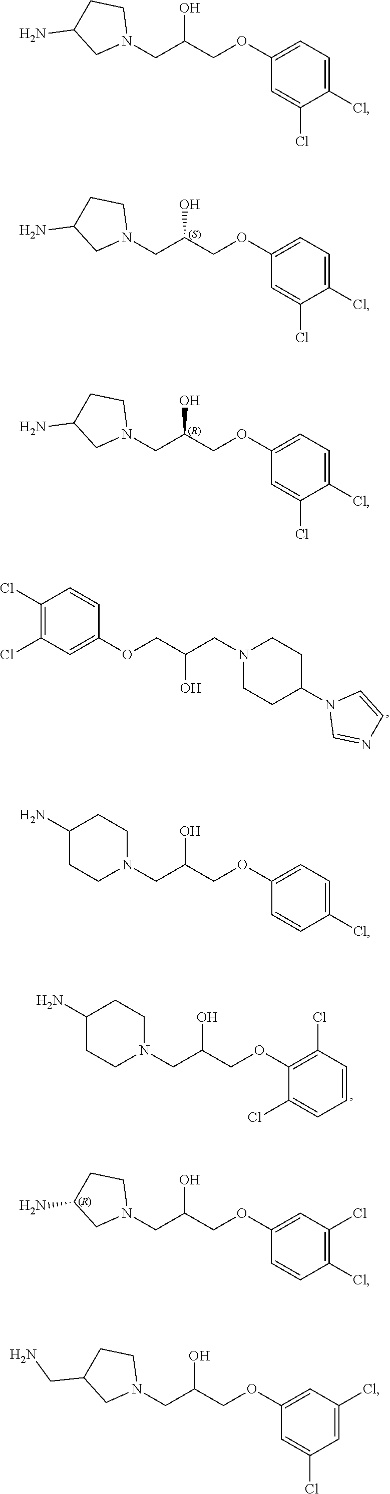 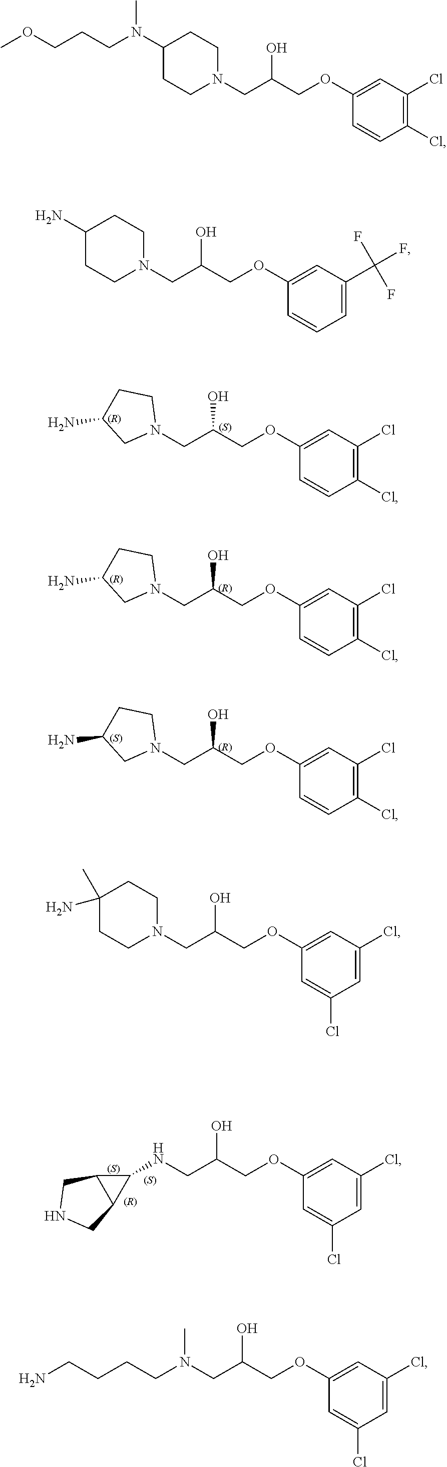 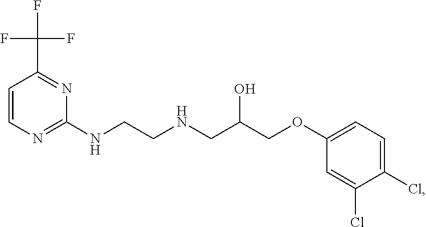 [SMILES: OC(CNCCNc1nccc(C(F)(F)F)n1)COc1ccc(Cl)c(Cl)c1]
or a pharmaceutically acceptable salt thereof.

40. The method of any one of claims 32-39, wherein the bacterial infection is resistant to one or more antibiotics.

41. The method of any one of claims 32-40, further comprising administering one or more antibiotics.

42. The method of claim 40, wherein the antibiotic is a macrolide, tetracycline, fluoroquinolone, penicillin, cephalosporin, aminoglycoside, sulfonamide, beta-lactam, tetracycline, trimethoprim-sulfamethoxazole, chloramphenicol, or lincosamide.

43. A method of treating a bacterial infection in a subject in need thereof, comprising administering to the subject a pharmaceutically acceptable amount of a compound of any one of claims 1-30 and one or more antibiotics.

44. The method of claim 43, wherein the bacterial infection is caused by an intracellular pathogen.

45. The method of claim 43, wherein the bacterial infection is caused by Gram-negative bacteria.

46. The method of claim 45, wherein the bacterial infection is caused by one or more Salmonella sp., Acinetobacter sp., Actinobacillus sp., Aeromonas sp., Bacteroide sp., Bordetella sp., Brucella sp., Burkholderia sp., Prevotella sp., Porphyromonas sp., Campylobacter sp., Citrobacter sp., Edwarsiella sp., Eikenella sp., Enterobacter sp., Escherichia sp., Francisella sp., Haemophilus sp., Helicobacter sp., Kingella sp., Klebsiella sp., Legionella sp., Moraxella sp., Morganella sp., Neisseria sp., Pasteurella sp., Plesiomonas sp., Proteus sp., Providencia sp., Pseudomonas sp., Salmonella sp., Serratia sp., Shigella sp., Stenotrophomonas sp., Streptobacillus sp., Vibrio sp., Yersinia sp., Chlamydophila sp., Ricketsia sp., Coxiella sp., Ehrlichia sp., or Bartonella sp.

47. The method of 46, wherein the bacterial infection is caused by one or more Salmonella species.

48. The method of claim 47, wherein the Salmonella sp. is S. enterica serovar Typhimurium.

49. The method of claim 45, wherein the bacterial infection is caused by E. coli, Klebsiella pneumonia, or Enterobacter cloacae.

50. The method of any one of claims 42-49, wherein the compound is: 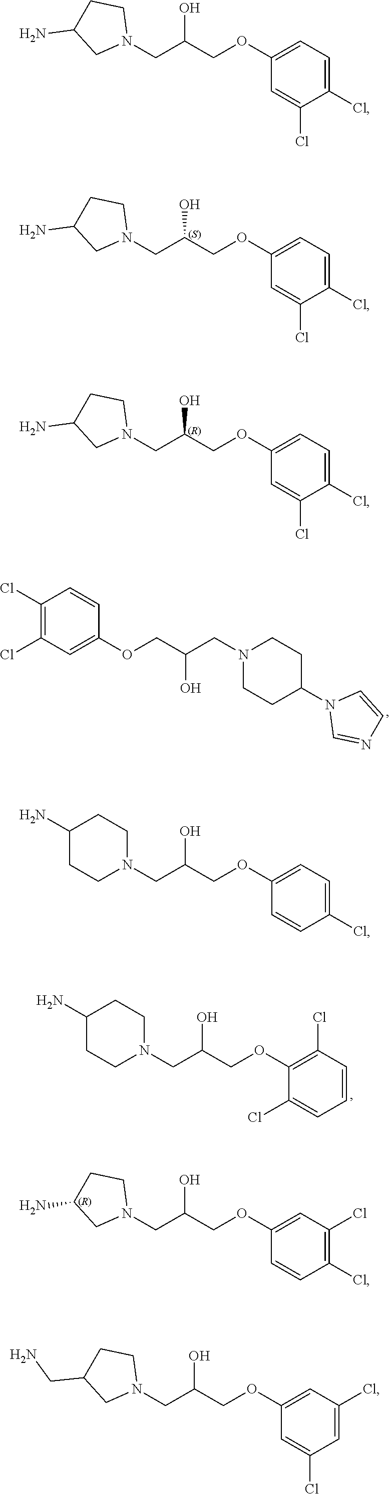 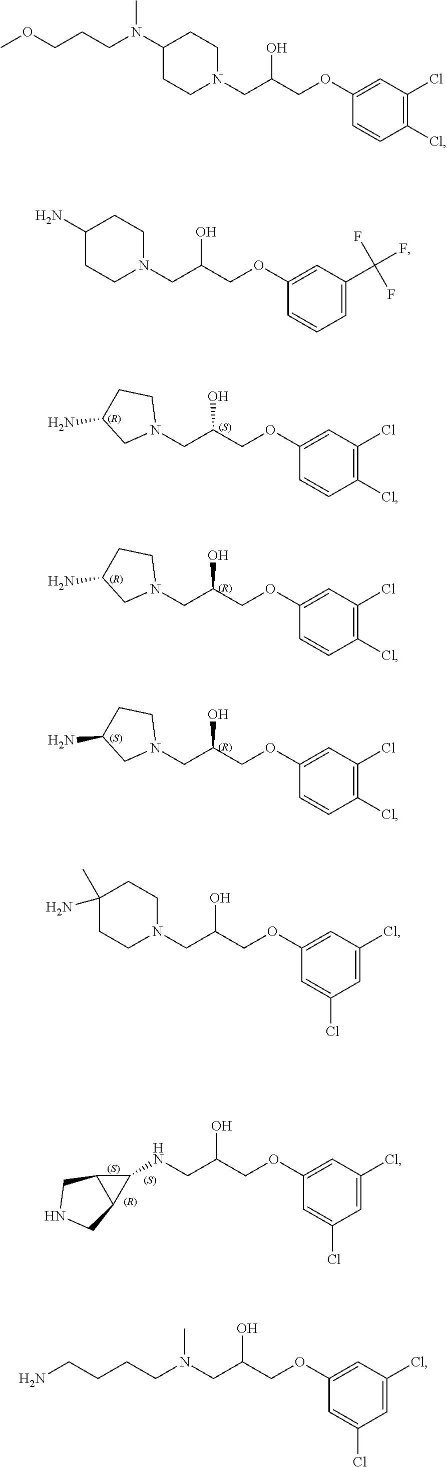 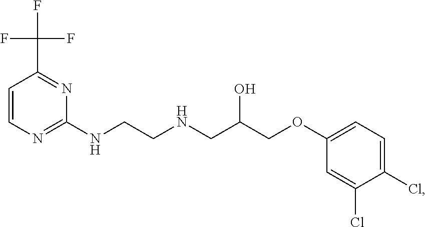 [SMILES: OC(CNCCNc1nccc(C(F)(F)F)n1)COc1ccc(Cl)c(Cl)c1]
or a pharmaceutically acceptable salt thereof.

51. The method of claim 42, wherein the antibiotic is a macrolide, tetracycline, fluoroquinolone, penicillin, cephalosporin, aminoglycoside, sulfonamide, beta-lactam, tetracycline, trimethoprim-sulfamethoxazole, chloramphenicol, or lincosamide.

52. A method of inhibiting bacterial efflux pump in a subject having a bacterial infection, comprising administering to the subject a pharmaceutically acceptable amount of a compound of any one of claims 1-30.

53. The method of claim 52, wherein the bacterial infection is caused by an intracellular pathogen.

54. The method of claim 53, wherein the bacterial infection is caused by Gram-negative bacteria.

55. The method of claim 54, wherein the bacterial infection is caused by one or more Salmonella sp., Acinetobacter sp., Actinobacillus sp., Aeromonas sp., Bacteroide sp., Bordetella sp., Brucella sp., Burkholderia sp., Prevotella sp., Porphyromonas sp., Campylobacter sp., Citrobacter sp., Edwarsiella sp., Eikenella sp., Enterobacter sp., Escherichia sp., Francisella sp., Haemophilus sp., Helicobacter sp., Kingella sp., Klebsiella sp., Legionella sp., Moraxella sp., Morganella sp., Neisseria sp., Pasteurella sp., Plesiomonas sp., Proteus sp., Providencia sp., Pseudomonas sp., Salmonella sp., Serratia sp., Shigella sp., Stenotrophomonas sp., Streptobacillus sp., Vibrio sp., Yersinia sp., Chlamydophila sp., Ricketsia sp., Coxiella sp., Ehrlichia sp., or Bartonella sp.

56. The method of claim 55, wherein the bacterial infection is caused by one or more Salmonella species.

57. The method of claim 56, wherein the Salmonella sp. is S. enterica serovar Typhimurium.

58. The method of any one of claims 52-57, wherein the compound is: 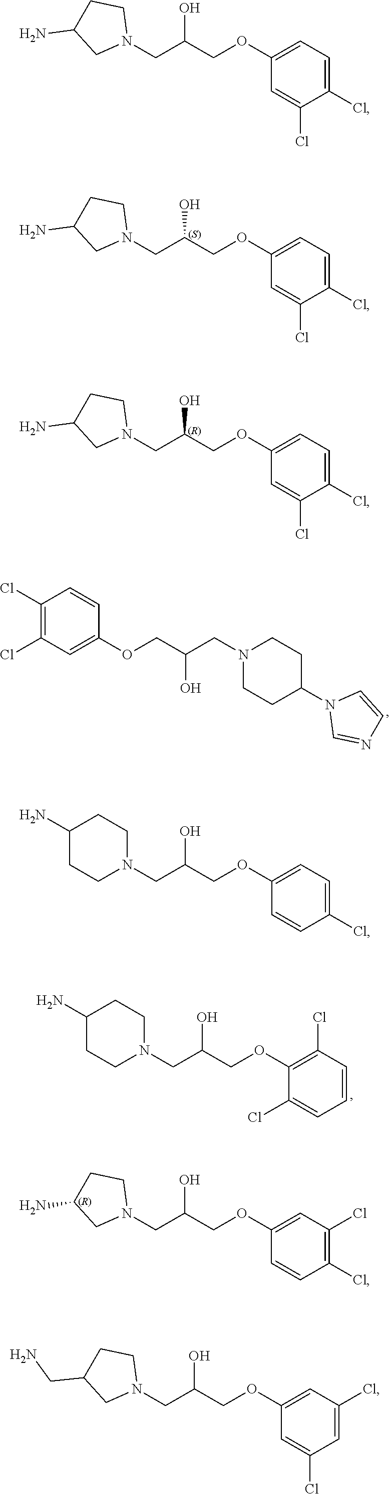 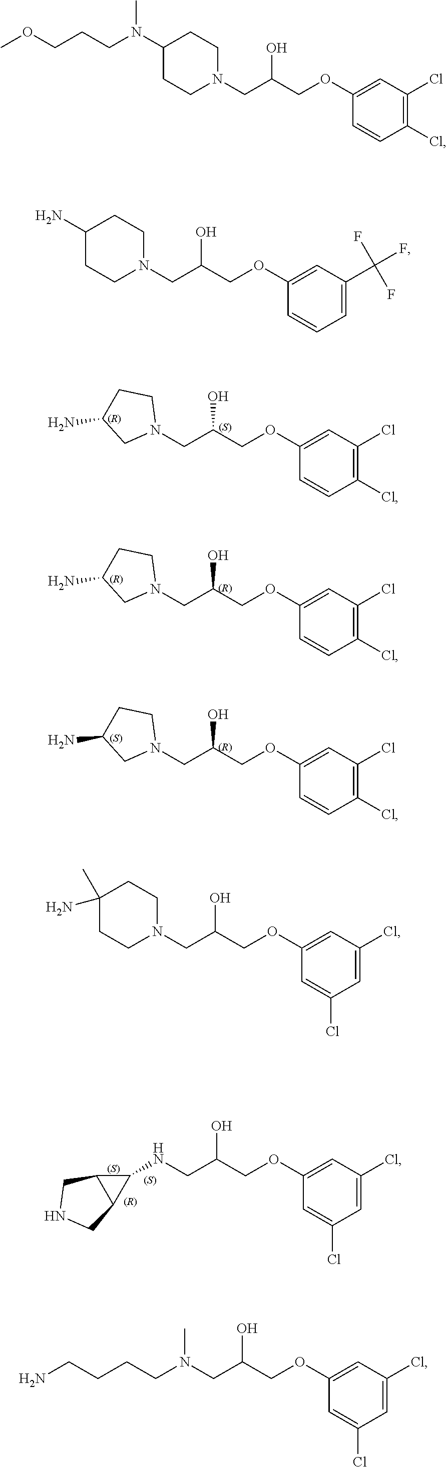 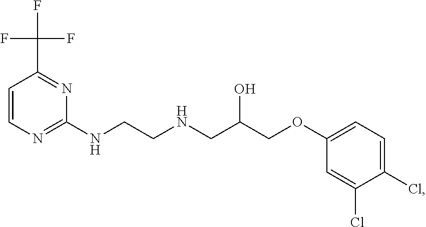 [SMILES: OC(CNCCNc1nccc(C(F)(F)F)n1)COc1ccc(Cl)c(Cl)c1]
or a pharmaceutically acceptable salt thereof.

59. The method of any one of claims 52-58, wherein the bacterial infection is resistant to one or more antibiotics.

60. The method of any one of claims 52-59, further comprising administering one or more antibiotics.

61. The method of claim 59, wherein the antibiotic is a macrolide, tetracycline, fluoroquinolone, penicillin, cephalosporin, aminoglycoside, sulfonamide, beta-lactam, tetracycline, trimethoprim-sulfamethoxazole, chloramphenicol, or lincosamide.

62. A method of increasing sensitivity of Gram-negative bacteria to an antibiotic, comprising administering a compound of any one of claims 1-30 in combination with an antibiotic.

63. A method of reversing or decreasing antibiotic resistance of an antibiotic-resistant Gram-negative bacteria, comprising administering a compound of any one of claims 1-30 in combination with an antibiotic.

64. The method of claim 62 or 63, wherein the antibiotic is a macrolide, tetracycline, fluoroquinolone, penicillin, cephalosporin, aminoglycoside, sulfonamide, beta-lactam, tetracycline, trimethoprim-sulfamethoxazole, chloramphenicol, or lincosamide.
